(19) United States
(12) Patent Application Publication
(10) Pub. No.: US 2025/0262354 A1
(43) Pub. Date: Aug. 21, 2025

(54) DECELLULARIZED TISSUE/POLYMER MULTI-COMPONENT BIOMATERIALS

(71) Applicant: YISSUM RESEARCH DEVELOPMENT COMPANY OF THE HEBREW UNIVERSITY OF JERUSALEM LTD, Jerusalem (IL)

(72) Inventors: Daniel COHN, Jerusalem (IL); Ariel ELYASHIV, Neve Daniel (IL); Abir KHALAILA, Sakhnin 4573 (IL); Aaron SLOUTSKI, Jerusalem (IL)

(21) Appl. No.: 18/252,484

(22) PCT Filed: Dec. 9, 2021
(86) PCT No.: PCT/IL2021/051465

§ 371 Date: May 10, 2023

Related U.S. Application Data
(60) Provisional application No. 63/123,817, filed on Dec. 10, 2020.
(60) Provisional application No. 63/131,101, filed on Dec. 28, 2020.

(51) Int. Cl. A61L 27/36; A61L 27/14; B32B 3/26; B32B 9/02; B32B 9/04; B32B 15/02; B32B 15/08; B32B 37/06

(52) U.S. Cl. CPC A61L 27/3691; A61L 27/14; A61L 27/3633; B32B 3/266; B32B 9/02; B32B 9/045; B32B 15/02; B32B 15/08; B32B 37/06; A61L 2300/606; B32B 2255/06; B32B 2255/26; B32B 2317/00; B32B 2535/00

(57) ABSTRACT

The technology concerns a construct comprising at least one tissular region and at least one polymeric region for use as an implant.

### TECHNOLOGICAL FIELD

The invention generally concerns a novel class of multi-component materials and uses thereof.

### BACKGROUND OF THE INVENTION

There is a wide variety of materials which are foreign to the human body and which are used in direct contact with its organs, tissues and fluids. These materials are called biomaterials, and among them, polymers play a pivotal role in all clinical areas, e.g., orthopedics, the cardiovascular arena, plastic surgery, drug delivery, wound dressings, among many others. Decellularized tissue is a unique class of biomaterials derived from living tissue. These materials play an important role in an increasing number of applications.

Polymers constitute one of the leading classes of materials used in implants for human or animal treatments due to the versatility of their chemical, physical, mechanical and biological properties and the extremely broad range of values they can attain. To illustrate this point, suffice to mention the following differences that pertain to their different properties: [i] chemically-polymers can be highly hydrophilic and water soluble as well as extremely hydrophobic materials, [ii] mechanically—their stiffness, as reflected by their respective Young's modulus values, ranges from a few kilopascals to hundreds of gigapascals, spanning over eight or more orders of magnitude, while their extensibility can be negligible or they can display strain at break values in the thousands percent range.

Decellularization (or acellularization) is a widely used technique to produce semi-natural biomaterials, whereby the cells and genetic materials are separated from the extracellular matrix (ECM) of the native tissue. Decellularization can be achieved by chemical, enzymatic or physical methods and is performed in such a way that the ECM retains its original chemical and structural properties. The resulting decellularized tissue, typically pericardium, omentum or small intestine mucosa, may be used in a number of areas, such as in hernia repair, for staple line reinforcement, in pelvic floor reconstruction, for dural closure, as a membrane in the dental arena, as cardiac patches or as leaflets of cardiac valves.

Pericardium, the collagen-rich membrane that encases the heart is the most widely used type of decellularized tissue and is currently utilized in various clinical applications. Among other indications, its use in dural closure, as bone membranes, as heart valves leaflets and for surgical buttressing are some of the more interesting. The most extensively used sources of decellularized pericardial tissue are bovine and porcine.

Despite their respective advantageous properties, both decellularized tissues and polymeric materials are limited in their behavior, especially in clinical scenarios, rendering their biological performance inadequate.

### SUMMARY OF THE INVENTION

The inventors of the technology disclosed herein have developed a new hybrid material that comprises a decellularized tissue and a polymeric material, wherein each is associated to other via one or more physical anchoring means. The interaction between the two components is not chemical in nature, while it is impossible to rule out random chemical interactions that may form between the tissue and the polymer.

Unlike known methodologies utilizing polymerizing reactive monomers, constructs of the invention are prepared by associating the decellularized tissue with a pre-made polymer. Such an assembly process overcomes the many drawbacks associated with the use of polymerizable monomers. These drawbacks may be related to [a] the monomers reactivity towards functional groups of proteins making up the decellularized tissue, affecting the tissue properties and biocompatibility, [b] monomers inherent toxicities, [c] monomers effective solubilization in organic agents, [d] monomers high volatility and flammability, [e] inability to position the monomers properly within the decellularized tissue, [f] undesired polymerization which may ensue due to a presence of certain functionalities that are present on the tissue and due to the catalyst/s used to trigger polymerization of the polymer component, [g] undesired polymerization within the tissue component, which can result in ill-defined polymers, varying in their average molecular weight and polydispersity and often their composition, [h] left over of residual monomers which can remain following polymerization and which may negatively affect the tissue and may also be extracted over time after being implanted, causing additional local as well as systemic problems.

By avoiding use of in situ polymerization of reactive monomers, the inventors have been able to achieve a stable and well defined construct which comprises at least one decellularized tissue and at least one polymeric component, wherein the polymeric component at least partially penetrates at least one surface region of the decellularized tissue.

Thus, in its broadest scope, the invention provides a construct comprising at least one decellularized tissue and at least one polymeric component, wherein the polymeric component at least partially penetrates at least one surface region of the decellularized tissue.

Putting it differently, the invention provides a decellularized tissue physically associated to a polymer component, said associating comprising or consisting at least partial penetration of the polymer component into a surface region of the tissue.

Also provided is a polymer-associated decellularized tissue, wherein association between the polymer and tissue is physical.

The invention further provides a construct of at least one decellularized tissue and at least one polymer, the construct being configured as an implant or a drug delivery device in vivo.

In another aspect, the invention provides a construct comprising at least one decellularized tissue and at least one polymeric component, wherein the polymeric component having at least one surface feature protruding one face of the decellularized tissue, crossing it to the other face through at least one hole formed in the tissue.

As used herein, the term “construct” is used to define a structure or an element or a device or an arrangement which comprises a tissular phase or region, in a form of at least one decellularized tissue, and a polymeric phase or region, in a form of at least one polymeric component, each as defined herein. The term does not suggest a particular arrangement or construction of the two phases or regions and in fact encompasses any arrangement whereby the two phases are present and associated to each other by means of a physical interaction. The term excludes such structures or elements whereby the association the no physical association exists between the two phases.

In all aspects of the invention, the polymeric components are implemented in a form of a sheet or a segment, i.e., a piece of polymeric material, such as a strip of the polymeric material, wherein the polymeric material is not formed in situ from monomers or prepolymers of the polymeric material. Constructs of the invention are formed, as will be further detailed herein, by using a premade polymer sheet or segment of a size and shape selected to meet a particular structure or use or may be formed on a surface region of the tissue from a solution or a liquid form of the polymer material. In other words, methods for preparing constructs of the invention are free of in situ polymerization steps utilizing monomers, oligomers or prepolymers.

The decellularized tissue and polymer may also be implemented in producing construct multisheets, which comprise two or more sheets of the tissue and a number of polymeric sheets or segments which hold the construct together. Multisheets of the invention may therefore be provided in a variety of forms, each having at least one tissue sheet and at least one polymeric sheet or segment. The sheets may be at least partially stacked on top of each other such that each of the polymer sheets or segments is associated to another polymer sheet in the stack, via at least one hole or pore formed in a decellularized tissue or via welding. While in the multisheet each of the material sheets is a solid sheet, one or more of the sheets positioned internally may be formed of a gel, a hydrogel or as a liquid or fluidic film.

As used herein, the term “sheet”, given its broadest meaning, is a continuous material or a spread of a material that may be in a form of a film of the material, of any size and shape and which typically consists of the polymeric material or the tissue. In constructs of the invention, where two material sheets are present, each sheet may be of the same or different material and may be of the same or different size and shape. In some embodiments, in multisheet constructs of the invention, each of the polymeric sheets may be the same or may be different in composition, structure, size, shape or any other physical, mechanical or chemical property.

Typically, sheets of the decellularized tissue are provided as elongated strips or ribbons of the tissue, the size and shape of which may vary. Similarly, the polymeric components may be provided as elongated sheets or strips of a size and/or shape that is similar or identical to that of the tissue, or may be provided as performed (or formed) segments or pieces or tags of the polymer which are of a size and shape different (typically smaller) than the tissue sheet.

In a multisheet construct of the invention are present at least one sheet of a decellularized tissue and at least one sheet or at least one segment of a polymeric component, wherein any sheet of the decellularized tissue is adjacent to or in contact with at least one sheet or segment of the polymeric component and wherein at least two sheets or segments of the polymeric components are associated to each other via at least one hole formed in the at least one sheet of the decellularized tissue.

In some embodiments, in a multisheet construct sheets or segments of the polymeric component are associated to each other via at least one hole formed in the at least one sheet of the decellularized tissue.

In some embodiments, in a multisheet construct at least two sheets or segments of the polymeric components are associated to each other by welding.

In some embodiments, at least one or any of the at least one sheets of a decellularized tissue is/are confined between any two sheets or segments of the polymeric component.

In some embodiments, the multisheet construct comprises a number of sheets of the decellularized tissue and the same number of sheets or segments of the polymeric component.

In some embodiments, the construct comprises a two or more assemblies of a decellularized tissue confined between two sheets or segments of a polymer component, wherein each of said assemblies is associated to each other, optionally by welding.

In some embodiments, at least two or any two assemblies are oppositely oriented.

The interaction or association between the polymeric component (sheet or segment) and the tissue is selected and configured to provide a robust anchoring of the polymer in the tissue, thus improving the construct mechanical properties. The improvement in the construct mechanical properties is achievable by a physical, non-chemical association that holds the two components together. The association, not being chemical in nature, may be defined as
(i) at least partial embedding of the polymeric sheet or segment in a surface region of a layer of the tissue, whereby the embedding may be through a single point on the surface region of the layer, or through two or more points, or
(ii) anchoring of the polymeric sheet or segment into the tissue to a tissue depth that permits secured association, whereby the anchoring may be through a single anchoring point, or through two or more anchoring points, whereby the depth of anchoring or penetration of the polymer sheet or segment may vary, not including puncturing of the tissue, or
(iii) anchoring the polymeric sheet or segment fully into the tissue to completely penetrate the tissue, from one face of the tissue to the other, whereby the anchoring may be through a single anchoring point, or through two or more anchoring points. Typically, such penetration would involve a surface feature that is configured to protrude one face of the decellularized tissue to the other through at least one hole formed in the tissue. As will be detailed below, such a feature may be formed in situ after holes are formed in the tissue or may be provided on the polymer sheet or segment in a form selected and configured to puncture or pierce the tissue.

As used herein, the expression “at least partially penetrate” a surface region of the decellularized tissue suggests any of the above interactions, ranging from embedding in a surface of the tissue, anchoring without piercing of the surface of the tissue to actual face to face penetrating of the tissue. Similarly, the expression “said associating comprising or consisting at least partial penetration of the polymer component” encompasses any of the above associations or interactions, suggesting a single type of association or interaction (consisting) or a combination of such associations or interactions (comprising).

The polymer sheets or segments are said to be associated to each other in a way that secures association with the decellularized tissue. One type of association or interaction present in constructs of the invention is via anchoring or piercing of the decellularized tissue surface, as detailed herein, or by forming holes in the tissue through which two polymer sheets or segments may be associates. In some embodiments of the invention, where the construct is made of a plurality of construct assemblies each assembly comprising e.g., a decellularized tissue confined between two sheets or segments of a polymer component, association of the plurality of assemblies may be achieved by polymer-to-polymer welding.

Generally speaking, welding of the polymers sheets of segments occurs when the polymer chains at the surface of one sheet or segment are mobile enough to entangle with chains in the other sheet or segment. To achieve welding, thermal energy may be applied to raise the temperature of the polymer above the appropriate transition temperature, i.e., the glass transition temperature, Tg, for amorphous thermoplastic polymers, or the melting temperature, Tm, for semi-crystalline polymers. When two sheets or segments of the polymeric component are brought into intimate contact under these conditions, polymer chain entanglement will occur resulting in a weld. According to aspects of the invention, welding need not be achieved over the full surface of the sheet or segment. Point welding at one or more regions of the polymeric component may suffice to provide a robust association of the plurality of assemblies or any two polymeric sheets or segments.

In some embodiments, the polymeric component is in a form of polymeric particles (nanoparticles, microparticles or particles of larger sizes) which are patterned on the surface of the tissue to form a particle sheet or particle continuity. In such embodiments, the particles may be embedded in the tissue surface (tissue outer layer), wherein the association between the tissue and the particles is strong enough to keep the association between the two over long periods of time.

In other embodiments, the association between the polymeric component and the tissue may involve at least one feature or functionality that is present on the polymeric component, e.g., as a pendent group (or as a ligand group present on the surface of the polymeric particle, or at least one polymeric material in a form of a layer or a sheet or a coat of particles or a polymeric sheet or a polymeric film or a polymeric fiber or a polymeric mesh), that penetrates or protrudes the tissue from one face thereof to the other. The penetration may be via pores existing in the tissue or via at least one hole that is pre-formed in the tissue e.g., by puncture, or by stamping, namely by placing the polymeric material in contact with the tissue and subsequently optionally applying pressure and/or temperature to induce physical penetration of the polymeric material into the tissue surface.

The at least one surface feature or functionality that penetrates or protrudes the tissue from one face thereof to the other may be in the form of elongated pins or needles that extend outwards from the surface of the polymeric material, and which are perpendicularly oriented to the surface of the polymeric material or are at an angle thereto. The size and shape of holes formed by the elongated pins or needles (i.e., size and shape of the pre-formed holes) may vary and the number or distribution of the holes may cover part or a complete surface of the decellularized tissue.

The pins or needles, or generally the at least one surface feature that is present on the surface of the polymeric material, may have an ending that secures the features in their place and prevents them from sliding out of the hole(s) formed in the tissue.

In some embodiments, the construct comprises a polymeric sheet having one or more tissue-penetrating features and a decellularized tissue decorated with one or more holes through which the features protrude, the tissue-penetrating features being composed of the material of the polymeric sheet. The patterning of the tissue penetrating features and/or the patterning of the holes may follow any patterning profile (hole profile), as defined herein (e.g., size, shape, density of distribution, position etc).

In some embodiments, the construct comprises two polymeric sheets or segments, each of which being associated to another via one or more polymeric feature or member extending a surface region of each of the sheets, wherein a decellularized tissue disposed between the two polymeric sheets having one or more holes through which said one or more polymeric members transverse.

In some embodiments, a construct is formed by piercing one or more holes in a surface region of the tissue and forming sheets or segments of the polymeric component by utilizing a liquid or fluid form of the polymeric component, wherein the liquid or fluid form is allowed or made to penetrate and fill up the holes.

In some embodiments, wherein two polymeric sheets or segments are present and one decellularized tissue disposed therebetween, each of the polymeric sheets is continuous and made of the same polymeric material, yet one of the sheets is larger in size as compared to the other.

In some embodiments, the construct is in a form of a multisheet device comprising one or more polymeric sheets and one or more sheets of decellularized tissue.

The invention also provides a multisheet construct comprising at least one sheet of a decellularized tissue and at least one sheet of a polymeric component, wherein the decellularized tissue being confined between any two sheets of the polymeric component and wherein any two sheets of the polymeric component are associated to each other via at least one hole formed in the at least one sheet of the decellularized tissue.

In some embodiments, one or more of the sheets may be designed as a material reservoir for releasing active or non-active materials, such as bioactives and drugs.

In some embodiments, at least a part of the polymer component is configured to release a material, such as a bioactive or a drug. In some embodiments, all or specific parts of the polymer component may locally release a bioactive or a drug.

The bioactives or drugs to be released from a construct of the invention following positioning in the body may be selected from any drug or pharmaceutical intended to achieve a medical improvement, prevent development of a diseases or a condition (locally or systemically), or sustain a state of good health over time. The bioactive or drug may therefore be selected based on, inter alia, the region of the body where the construct is to be implanted or positioned and the types of medical complications that may be associated with the site of implantation and the procedure.

Generally speaking, the actives or drugs are broadly characterized as non-toxic and regulated by the FDA or EMA or classified as GRAS (Generally Recognized As Safe). Non-limiting examples of such actives and drugs may include analgesics including non-narcotic and narcotic analgesics; antianxiety drugs; antiarrhythmics; antibacterial agents; antibiotics including naturally occurring, synthetic, broad-spectrum antibiotics; anticoagulants and thrombolytics for arterial or venous thrombosis; anticonvulsants; antidepressants including mood-lifting antidepressants: tricyclics, monoamine oxidase inhibitors, and SSRIs; antidiarrheals including antidiarrheal preparations and drugs that slow down the contractions of the bowel muscles; antiemetics; antifungals including infections that affect hair, skin, nails, mucous membranes; antihistamines; Antihypertensives including diuretics, beta-blockers, calcium channel blocker, ACE (angiotensin-converting enzyme) inhibitors; anti-inflammatories; antineoplastics; antipsychotics including major tranquilizers; antipyretics; antivirals including treatment and temporary protection against viral infections; beta-blockers; corticosteroids in the context of immunosuppression, malignancies or deficiency disorders; cytotoxics as antineoplastics and also as immunosuppressives; hormones including synthetic equivalents and natural hormone extracts; immunosuppressives; muscle Relaxants including those that relieve muscle spasm and minor tranquilizers; sex hormones (Female) including those used for menstrual and menopausal disorders, oral contraceptives, and also for treating female and male cancers; sex hormones (Male) including those used for male hormonal deficiency in hypopituitarism or disorders of the testes, also for treating cancer, and anabolic steroids; enzymes such as collagenase or elastase; and vitamins.

In some embodiments, any of the polymeric or tissue components may be porous, namely including surface pores which are confined to a particular region of the component or may be distributed along the whole surface of the polymer or tissue. The pores may be inherently present or formed in the decellularized tissue to achieve a particular hole (or porosity) profile. The profile defines the at least one surface region of the tissue where holes are to be formed; the number, shape and size of the holes; the density of holes at a certain surface region, etc. Different patterning profiles may be utilized to meet a desired construct capabilities or attributes or utility. Once one or more holes are formed, the association of the polymeric material may proceed by a variety of methodologies, as disclosed herein. Some may involve use a polymeric component having protruding features that can be inserted or penetrated through the holes. Other methodologies may involve use of a liquid polymer. In such methodologies, the tissue may be immersed, sprayed or generally treated in or with the liquid polymeric material or a solution comprising the polymeric material, allowing said polymeric material to penetrate the hole(s) and further deposit on the tissue faces thereby forming a polymeric sheet or segment on either or both tissue faces. The polymeric sheets or segments formed on either or both faces of the tissue may be fused or welded to another polymeric component or sheet to thereby, layer by layer, or component by component establish a multisheet or construct of the invention.

Alternatively, decellularized tissues are pierced and a liquid polymer is injected into holes created in the tissues. The assembly is constructed by forming a polymer sheet between two tissue sheets and construct is thereafter thermally bonded. The construct may be attached to another polymer sheet or feature by welding.

According to yet another approach, a multisheet structure is formed by stacking the various sheets on top of each other, the multisheet is thereafter pierced to afford a desired hole profile and injected with a liquid polymer to afford the fused construct.

According to yet another approach, liquid polymer is cast onto a tissue to form a bilayer of tissue and polymer. Subsequently, the bilayer is pierced to provide a hole profile and injected with a liquid polymer to afford the construct. The binary construct may be used to form a multisheet or may be welded to another polymer sheet.

In some embodiments, in a construct of the invention, the decellularized tissue constitutes between 5% wt and 95% wt of the construct.

As used herein, the “decellularized tissue” is a tissue from which inhibiting cells have been removed, leaving behind the extracellular matrix (ECM) of a tissue. As noted above, decellularization can be achieved by chemical, enzymatic or physical methods, as known in the art. The decellularized tissue may be obtained from a tissue of the oral mucosa, from the small intestinal submucosa or from bladder-decellularized matrixes, which offer natural and optimal integration properties to the extracellular matrix. Other tissues may also be used.

Methods for achieving a decellularized tissue are known in the art, for example in International Application No. WO2005/032473 and US applications derived therefrom each being incorporated herein by reference; U.S. Pat. No. 5,993,844, herein incorporated by reference; and others.

In some embodiments, the decellularized tissue is selected from pericardium, omentum or small intestine mucosa.

In some embodiments, the decellularized tissue is pericardium.

In some embodiments, the decellularized tissue is a bovine pericardium or a swine pericardium.

In some embodiments, the decellularized tissue is a small intestinal submucosa.

In some embodiments, the decellularized tissue is omentum.

The polymer used in a construct of the invention is a polymeric material as known in the art. The polymer used may be selected amongst thermoplastic polymers and thermoset polymers. The polymer may be hydrophobic, hydrophilic, or amphiphilic and may be further selected amongst bioinert polymers or biodegradable polymers.

In some embodiments, the polymer is a blend of different polymers, an interpenetrating polymer network (IPN), or a semi-interpenetrating polymer network (semi-IPN).

In some embodiments, the semi-IPN polymers are selected to crosslink via addition or condensation reactions, click chemistry reactions or any other type of reaction and combinations thereof. In some embodiments, the compounds capable of crosslinking are selected from compounds containing two or more carbon double bonds, such as ethylene glycol dimethylacrylate (EGDMA) and ethylene glycol diacrylate (EGDA), triethylene glycol dimethacrylate (TEGDMA), tetra(ethylene glycol) diacrylate (TEGDA), divinylbenzene (DVB), bis-acrylamide, polyethylene glycol dimethacrylate (PEGDMA), polyethylene glycol diacrylate (PEGDA), polypropylene glycol dimethacrylate (PPGDMA), polypropylene glycol diacrylate (PPGDA), polyethylene glycol/polypropylene glycol (PEG/PPG), copolymeric dimethacrylate (DMA) and diacrylate (DA), polytetramethylene glycol dimethacrylate (PTMGDMA) and polytetramethylene glycol diacrylate (PTMGDA, polydimethylsiloxane DMA and DA, polycaprolactone dimethacrylate (PCLDMA and polycaprolactone diacrylate (PCLDA), polycaprolactone/lactide P(CL/LA), DMA and DA, polycaprolactone/glycolide P(CL/GA), DMA and DA and polyglycolide/lactide P(GA/LA), DMA and DA, polyethylene glycol/caprolactone (PEG/CL), DMA and DA, polyethylene glycol/lactide PEG/LA, DMA and DA and polyethylene glycol/glycolide (PEG/GA) DMA and DA, polypropylene glycol/caprolactone (PPG/CL) DMA and DA, polypropylene glycol/lactide (PPG/LA), DMA and DA and polypropylene glycol/glycolide (PPG/GA) DMA and DA, polytetramethylene/caprolactone (PTMG/CL), DMA and DA, polytetramethylene/lactide (PTMG/LA), DMA and DA and polytetramethylene/glycolide (PTMG/GA), DMA and DA.

In some embodiments, the IPN consists of the polymers formed by compounds containing two or more carbon double bonds selected from a group consisting of EGDMA and EGDA, TEGDMA and TEGDA, DVB, bis-acrylamide, PEG DMA and DA and higher functionalities of various molecular weights, PPG DMA and DA and higher functionalities of various molecular weights, PEG/PPG copolymeric DMA and DA and higher functionalities, PTMG DMA and DA and higher functionalities of various molecular weights, siloxane DMA and DA and higher functionalities of various molecular weights, PCL DMA and DA and higher functionalities of various molecular weights, P(CL/LA), P(CL/GA) and P(GA/LA) DMA and DA and higher functionalities of various molecular weights, PEG/CL, PEG/LA and PEG/GA DMA and DA higher functionalities of various molecular weights, PPG/CL, PPG/LA and PPG/GA DMA and DA higher functionalities of various molecular weights, PTMG/CL, PTMG/LA and PTMG/GA DMA and DA higher functionalities of various molecular weights, and combinations thereof.

In some embodiments, the polymer is an acrylic or a methacrylic polymer.

In some embodiments, the polymer is a polyolefin.

In some embodiments, the polymer is a silicone polymer.

In some embodiments, the polymer is a polycarbonate, a polyurethane, a polyurea or a polyamide and combinations thereof. In some embodiments, the polymer is a polyurethane.

In some embodiments, the polymer has a glass transition or a melting point below 120° C. In some embodiments, the polymer has a glass transition or a melting point below 85° C.

In some embodiments, the polymer is selected to flow under pressure of 50 kPa and a temperature above 42° C.

In some embodiments, the polymer is selected from polymethyl methacrylate (PMMA), poly(n-butyl methacrylate) (PBMA), poly(hexyl methacrylate) (PHMA), polystyrene (PST), poly(2-hydroxyethyl methacrylate) (PHEMA), poly(N-(2-hydroxypropyl)methacrylamide) (PHPMA), polycyanoacrylate (PCA), a polyethylene/polypropylene copolymer, a polyethylene/polybutylene copolymer, a polypropylene/polybutylene copolymer, poly-isobulyene, polydimethylsiloxane (PDMS), phenyl-containing PDMS, polyester urethanes, polyether urethanes (e.g. Pellethane, Elastane, Elastolan, Tecoflex, Biomer), polycarbonate urethanes (e.g. Chronoflex, Biospan and Bionate) and silicone-containing polyurethanes (e.g. CarboSil, PurSil, Avcothane and Cardiothane), polyglycolic acid, polylactic acid, polycaprolactone, polylactide-caprolactone copolymer, polyglycolic acid-lactic acid copolymer, polyethylene oxide-polylactic acid copolymer, polyethylene oxide-polycaprolactone copolymer, polytetramethylene oxide-caprolactone copolymer, polyhydroxy butyrate, polyhydroxy valerate, polyethylene adipate, polybutylene adipate, polyethylene succinate polybutylene succinate and polybutylene terephthalate and polyethylene/butylene terephthalate copolymers and combinations and copolymers thereof.

In some embodiments, the polymer is an aliphatic polyether-based thermoplastic polyurethane, being optionally Tecoflex.

In some embodiments, the polymer is an aromatic polycarbonate-based urethanes, being optionally Chronoflex.

In some embodiments, the polymer is PMMA, PBMA, PHMA, PMA, PHEMA, PHPMA and combinations thereof.

In some embodiments, the polymer is a low molecular weight (typically between 500 and 10,000 Da) or amorphous or branched polyolefin and combinations thereof.

In some embodiments, the polymer is PDMS or a phenyl containing PDMS or derivatized PDMS chains containing double bonds and/or hydroxyl and/or amine and/or thiol groups.

In some embodiments, the polymer is an aliphatic or aromatic polyurethane comprising a polyether or polyester soft segment.

The polymer may be selected to have a morphology and properties that change over time after. These changes may be due to chemical, physical and/or biological phenomena. In some embodiments, the polymer is selected to undergo secondary chemical changes such as coupling, polymerization or crosslinking, oxidation or hydrolytic or enzymatic degradation. In some embodiments, separately or in addition to the chemical changes, the polymer may be selected to undergo or be affected by physical processes, such as crystallization or phase separation.

In some embodiments, the polymer is a shape-memory-displaying polymer that can be actuated by various stimuli such as temperature, pH, ionic strength, hydration, biological cues, an electric or magnetic field, radiation of any type and combinations thereof, wherein shape memory displaying polymer can display a onetime response or a cyclic shape memory response.

In some embodiments, the actuated shape memory polymer is a polymer capable of modulating a shape of the tissue/polymer construct.

In some embodiments, all or part of the polymer component may display shape memory behavior.

In some embodiments, all or part of the polymer component may be environmentally responsive. In some embodiments, all or part of the polymer component may be reverse thermo-responsive.

In some embodiments, the polymer is an environmentally responsive polymer that is responsive to temperature, pH, ionic strength, biological cues, radiation of various types, an electric or magnetic field, and combinations thereof. In some embodiments, the environmentally responsive polymer is reverse thermo-responsive.

The reverse thermo-responsive may be selected from N-alkyl substituted acrylamides (such as poly-N-isopropyl acrylamide [PNIPAAm]) or is based on polyethylene oxide/polypropylene oxide segments, or is a cellulose derivative selected from hydroxypropyl methylcellulose and hydroxypropyl cellulose, or an alternating or random polymer, and various amphiphilic polymers such as poly(ethylene oxide)-polylactic acid block copolymers.

In some embodiments, polymer is polybutyl methacrylate.

In some embodiments, the polymer is a polyolefin such as a polyethylene/polypropylene copolymer, a polyethylene/polybutylene copolymer, a polypropylene/polybutylene copolymer, poly-isobulyene or combinations thereof.

In some embodiments, the polymer is a silicone polymer that is a derivatized PDMS, selected to crosslink in situ.

In some embodiments, the polymer is a polyurethane selected from Pellethane, Elasthane, Elastolan, chronoflex, Tecoflex, Cardiothane, Avcothane, CarboSil, PurSil, Biomer, BioSpan and Bionate, each as defined herein, and combinations thereof.

In some embodiments, the polyurethane is an aliphatic polyether-based thermoplastic polyurethane, e.g., Tecoflex.

In some embodiments, the polyurethane is an aromatic polycarbonate-based urethanes, e.g., Chronoflex.

In some embodiments, the polymer is a polyester and is optionally selected from polyglycolic acid, polylactic acid, polycaprolactone, polyhydroxy butyrate, polyhydroxy valerate, polyethylene adipate, polybutylene adipate, polyethylene succinate polybutylene succinate and polybutylene terephthalate and polyethylene/butylene terephthalate copolymers and combinations and copolymers thereof.

In a construct of the invention, the polymer used may be a blend or a mixture or a combination of two or more polymeric materials. In some embodiments, such a blend may comprise two or more polyolefins or acrylic or methacrylic polymers or silicone polymers or polycarbonates or polyurethanes or polyureas or polyamides and combinations thereof.

In another aspect, a construct of the invention comprises one or more tissular phase (comprising or consisting the decellularized tissue) and more than one polymeric phase (comprising or consisting the polymer), each of which forming a separated phase of different geometries, and the size of which can span from the nanometric to the centimetric scale.

The polymeric phase and/or the tissular phase may be constructed or composed by more than one type of polymers or decellularized tissue. Each of the polymers and/or tissues may form a single construct or form an array of constructs that are associated to each other, as further disclosed herein. In some embodiments, a polymer and/or a tissue may be part of a medical device having at least one polymeric region and/or at least one tissular region and the construct may be associated thereto. Such a device may be comprised of materials of all types, for example tissues, polymers, metals, ceramics, carbonaceous materials and combinations thereof.

In a construct of the invention, the polymeric phase is configured or selected or engineered to connect to two or more of the tissue phases or other phases comprising materials, as disclosed above, for example polymers, metals, ceramics, carbonaceous materials and combinations thereof. In some embodiments, the polymeric phase is configured to connecting two or more of the tissue phases or other phases and said connection can sustain a stress that is above 5% of the cohesive strength of the polymer itself. In some embodiments, for these and other purposes, polymer is thus selected to display shape memory behavior. In some embodiments, the polymer is environmentally responsive. In some embodiments, the polymer is biodegradable.

In some embodiments, the polymer is reverse thermo-responsive and forms aqueous solutions undergoing an LCST transition below 37° C.

In some embodiments, the construct is a composite material.

The invention further provides an implant or a medical device being or comprising a construct of the invention.

Irrespective of the type of construct used, in accordance with the invention, and irrespective of the sort of device to be manufactured from a construct of the invention, the construct is configured or engineered or intended for coming into contact with human or animal tissues or organs. In some cases, the construct is a device that is intended for association with a tissue in the human or animal body or may be structured as a device to be implanted in the body. For example, the construct may constitute a medical device or be part of a medical device configured for implanting in a subject's GI tract, in the respiratory system (airway system), along the vasculature, in the cardiac arena, in the urinary system, or in any other organ of the human or animal body, as may be the case. Depending, inter alia, on the construct materials, shape and intended use, and further on the organ where the implant is to be used, the construct may be porous or non-porous, where said pores can span from the nanometric to the centimetric scale. Non-limiting examples of devices making use or comprising a construct of the invention include stents, metallic stents, vascular grafts, heart valves (comprising optionally a metallic frame), membrane, sealing devices, suture or staple lines, hernia meshes or hernia repair devices, pelvic floor reconstruction devices, wound or burn dressings, dural closures, cardiac patches and others.

In some embodiments, the device is implemented in a heart valve comprising also a metallic frame. The heart valve may thus comprise, in some embodiments, pericardium leaflets, a tissue/polymer phase and a metallic frame, as defined herein.

In some embodiments, the construct is a patch deployed in the CV system, along the GI tract, in the airway tree, in the urinary and reproductive arena, in the central or peripheral nerve system, a vascular graft, an A/V shunt, wherein said polymer/s form/s a film, a fiber, particles of any size and geometry, porous or solid, hollow or not, or any other geometry, produced by any manufacturing techniques, including textile methods and procedures producing non-woven structures and combinations thereof Devices of the invention, amongst which the construct of the invention is included, may be used as vehicles for delivery of one or more active agents to a site or an organ or a tissue in a subject's body. Such active agents may be cellular or molecular bioactive materials. The active material may be contained in the construct as a whole, in the polymer component or in the decellularized tissue. In some embodiments, the decellularized tissue contains an active, e.g., a bioactive material, as defined herein. In some embodiments, the active material is released over time.

Constructs of the invention may be provided in a variety of forms, as exemplified herein. In some embodiments, the polymer is associated to two or more tissue regions or segments, thus forming a laminate or any other structure such as a multilayered structure. In some embodiments, a multilayered structure may comprise three layers of the polymer, two of which being external and one in the middle of said construct with two layers of a tissue sandwiched between the polymer layers.

In some embodiments, the construct is provided with a polymeric film confined between two sheets of the decellularized tissue.

In some embodiments, the construct may be formed with polymeric connections that associate each of the polymer layers via holes made in the tissue layers. In some embodiments, the polymeric connections have the same or a different composition than the polymers forming the layers. To synthesize such a construct, according to the present invention, a polymer or polymeric mixture used and pre-formed, e.g., into a polymer film, is selected to be able to flow under a pressure between 50 kPa and 1 MPa and a temperature below 120° C., filling holes made in a tissue, thereby connecting or associating the tissue and polymer together. Once cooled down, the polymer generates a continuous film-like structure distally to the direction of flow, contributing to the strength and long-term stability of the association between the tissue and polymer phases.

In some embodiments, the polymer is an RTR polymer which flows under a pressure below 100 kPa and a temperature below 37° C. Once at physiological temperature, the polymer generates a continuous film-like structure distally to the direction of flow, contributing to the strength and long-term stability of the association between the tissue and polymer phases.

In some embodiments, the polymer in a construct of the invention is pre-formed, optionally in the form or shape of a film. The pre-formed polymer may be comprised of more than one material, blended together or separated in space at distances spanning from the nanometric to the centimetric scale, organized isotropically or anisotropically, generating layers or in any other spatial arrangement, wherein said additional materials can be selected from polymers, tissues, active components, metallics, ceramics or carbonaceous materials and combinations thereof.

In some embodiments, the pre-formed polymer further comprises a low molecular weight molecule that softens the film or parts thereof. The low molecular weight molecule enables the polymeric phase or part of it to flow under pressure and temperature conditions. In some embodiments, the low molecular weight molecule is polymerizable or crosslinkable, so it softens the polymer phase or parts of it before it polymerizes or crosslinks and strengthens or stiffens said polymer phase once polymerized or crosslinked. In some embodiments, said low molecular weight molecule, inert or reactive, may be bioinert or biodegradable, may display shape memory behavior or may be environmentally responsive, and combinations thereof.

In some cases, the construct is obtainable by polymerization or crosslinking reactions that take place via addition or condensation reactions or any other type of reaction and combinations thereof. The addition polymerization or crosslinking reactions may involve reactive carbon double bonds following any type of catalysis, including chemically-, thermally- and radiation-initiated reactions.

In some cases, the construct is obtainable by polymerization or crosslinking reactions of suitable precursors wherein said reactions are conducted partially before being in contact with the decellularized tissue and partially during or following the association of the polymeric component with the decellularized tissue component. In some embodiments, the partially polymerized or crosslinked polymer film or sheet or construct having any geometry can flow under the conditions applied, generating the desired association with the decellularized tissue or any other component, polymeric or not. In some embodiments, the partially polymerized or crosslinked polymer is selected to flow under pressure of 50 kPa and a temperature above 42° C.

In some embodiments, the partially polymerized or crosslinked polymer has a glass transition or a melting point below 120° C., while in some other embodiments, the partially polymerized or crosslinked polymer has a glass transition or a melting point below 85° C.

In other embodiments, the partially polymerized or crosslinked polymer has a glass transition or a melting point below 60° C.

In some embodiments, the partially polymerized or crosslinked polymer that is further polymerized or crosslinked during or following association with the decellularized tissue or any other component, polymer or not, tissular or not, has a higher glass transition or a melting point than the partially polymerized or crosslinked polymer.

In some embodiments, the further polymerized or crosslinked polymeric member has a glass transition or a melting point above 50° C. and in other embodiments the further polymerized or crosslinked polymeric member has a glass transition or a melting point above 60° C. and yet in other embodiments the further polymerized or crosslinked polymeric member has a glass transition or a melting point above 80° C.

In some embodiments, the further polymerized or crosslinked polymeric member has a glass transition or a melting point above 100° C.

In some embodiments, the polymer in a construct of the invention is pre-formed, optionally in the form or shape of a film and comprises partially polymerized or crosslinked polymer components differing in the extent of their initial degree of polymerization and/or crosslinking and in the final degree of polymerization and/or crosslinking attained when further polymerized or crosslinked polymeric member.

In some embodiments, the pre-formed polymer further comprises a low molecular weight molecule (between 500 and 100,000 DA), polymerizable and/or crosslinkable or not, and partially polymerized or crosslinked suitable precursors wherein said polymerization or crosslinking reactions are conducted partially before being in contact with the decellularized tissue and partially during or following the association of the polymeric component with the decellularized tissue component.

In other cases, the construct may be obtainable through reaction of compounds selected from methyl methacrylate (MMA), butyl methacrylate (BMA), Hexyl MA, styrene (ST), (2-hydroxyethyl methacrylate) (HEMA), acrylamide (AAm), acrylic acid (AAc), N-vinyl pyrrolidone (NVP), cyanoacrylates, N-iso-PAAm, Maleic anhydride, EGDMA and EGDA, TEGDMA and TEGDA, DVB, bis-acrylamide, PEG DMA and DA and higher functionalities of various molecular weights, PPG DMA and DA and higher functionalities of various molecular weights, PEG/PPG copolymeric DMA and DA and higher functionalities, PTMG DMA and DA and higher functionalities of various molecular weights, siloxane DMA and DA and higher functionalities of various molecular weights, PCL DMA and DA and higher functionalities of various molecular weights, P(CL/LA), P(CL/GA) and P(GA/LA) DMA and DA and higher functionalities of various molecular weights, PEG/CL, PEG/LA and PEG/GA DMA and DA higher functionalities of various molecular weights, PPG/CL, PPG/LA and PPG/GA DMA and DA higher functionalities of various molecular weights, PTMG/CL, PTMG/LA and PTMG/GA DMA and DA higher functionalities of various molecular weights, and combinations thereof.

The reactions may be selected from click chemistry reaction, complexation of any type, host/guest reactions and non-covalent supramolecular polymerizations. In some embodiments, condensation reactions are involved whereby urethane, urea, amide, ester, carbonate, or ether groups and combinations thereof are formed.

In some embodiments, epoxy/amine reactions and hydrolysis and condensation reactions of siloxane containing molecules may be involved. In some embodiments, the reaction may involve reactions of thiol groups. The epoxy/amine reactions may take place between GMA and amines of various functionalities and molecular weights, including amino-terminated PEG, PPG, PTMG, PCL, PDMS, containing one or more amino groups per molecule, amino-terminated P(CL/LA), P(CL/GA) and P(GA/LA) containing molecules of various molecular weights, PEG/CL, PEG/LA and PEG/GA containing molecules of various molecular weights, PPG/CL, PPG/LA and PPG/GA containing molecules of various molecular weights, PTMG/CL, PTMG/LA and PTMG/GA containing molecules of various molecular weights containing one or more amine groups, oligopeptides and peptides containing reactive amine moieties, and epoxy-terminated molecules of various functionalities and molecular weights able to react with said amino-terminated molecules, and combinations thereof.

In some embodiments, molecules of the polymeric material, in a construct of the invention, are able to disentangle, to cross the polymer/tissue interphase or any other interphase, to diffuse into the tissue phase or any other phase, intermingling and re-entangling with themselves and with the molecules of the tissue or any other phase, welding the two together. In some embodiments, the molecules that crossed the polymer/tissue interface are capable of also reacting with themselves and/or with moieties present in the tissue or any other phase, enhancing the strength and long-term stability of the connection between the polymer phase and any other phase.

The construct of the invention may be formed wherein the polymeric material is generated while in a liquid or semi-liquid state, in direct contact the tissue phase (in situ). The in situ generated polymeric phase is formed by applying a liquid or semi-liquid polymerizable or crosslinkable reactive precursor of optimized composition. In some embodiments, said in situ generated polymeric phase is formed by applying a solution of said polymer on the surface of the tissue phase and/or within the tissue phase, and combinations thereof. In some embodiments, the polymer solution is an aqueous solution or a solution using other hydrophilic solvents. In some embodiments, the polymer is dissolved in an organic solvent, such as acetone, THF, dioxane, DMSO, halogenated solvents such as chloroform and dichloromethane, alcohols and polyols, polyethers, acetonitrile and ethyl acetate. The polymer solution has a concentration ranging from 1 wt % to 40 wt %, or from 1 wt % to 20 wt % or from 2 wt % to 10 wt %.

In some embodiments, the tissue and polymer phases are connected by applying pressure, at a supra-physiological temperature for a period shorter than one hour. In some embodiments, the pressure ranges from 20 kPa to 100 GPa, and the temperature spans from 40 degrees up to 120 degrees, applied for a time interval from 1 second to 60 minutes. In some embodiments, the pressure ranges from 500 kPa to 10 GPa, and the temperature spans from 40 degrees up to 85 degrees, applied for a time interval from 2 seconds to 5 minutes.

Thus, in other aspects of the invention, there are provided methods of manufacturing constructs of the invention.

According to one aspect, a process is provided for manufacturing a construct according to the invention, the process comprising
contacting a pierced surface region of at least one decellularized tissue with a liquid polymer, and
permitting said liquid polymer to penetrate into the piercings (holes) and form a polymeric sheet on the surface region.

The pierced surface region of the tissue may be one which holes have been made there through or which naturally comprises such holes or pores. Where holes are to be made, they may be made by using a cutting or piercing device, such as a needle of various diameters. The position of the holes, shape of holes, their size and their distribution over the surface region (hole profile, as defined herein) may be predefined and selected to meet any prerequisites relating to, e.g., the mechanical properties of the construct or to its use. Typically, the number of holes is greater than 1.

Thus, in some embodiments, the process comprises piercing or forming holes in a surface region of at least one decellularized tissue. In some embodiments, the holes have a predefined hole profile, as defined.

In some embodiments, the liquid polymer is injected into the piercings (holes).

When the liquid polymer is used, curing, e.g., by thermally treating the polymer, may be required. As different polymers may be used, different curing methodologies may be used.

A further process is provided for manufacturing a construct comprising at least one decellularized tissue and at least one polymeric component, wherein the process comprises
contacting a surface region of at least one decellularized tissue having been pierced to form one or more holes with a liquid polymer and
permitting said liquid polymer to penetrate into the one or more holes and form a polymeric sheet on the surface region.

In some embodiments, the process comprises piercing or forming holes in a surface region of at least one decellularized tissue. In some embodiments, the holes have a predefined hole profile, as defined.

In some embodiments, the liquid polymer is injected into the piercings (holes).

In some embodiments, the liquid polymer fully penetrates through the one or more holes.

In some embodiments, the liquid polymer partially penetrates the one or more holes.

In some embodiments, the liquid polymer fully penetrates through the one or more holes to form a polymer sheet on both faces of the surface region, to thereby form a construct assembly.

In some embodiments, the process comprises a step of associating or fusing two or more constructs. Fusion may be by welding.

Further provided is a process for manufacturing a construct comprising at least one decellularized tissue and at least one polymeric component, the process comprising
stacking one or more sheets of a decellularized tissue and one or more sheets or segments of a polymeric material to obtain a stacked structure;
forming holes in said stacked structure to form one or more holes in each of the one or more sheets of the tissue and polymeric material, wherein optionally at least a number of said one or more holes are coaxially arranged; and
treating said stacked structure with a liquid polymer to cause said liquid polymer to penetrate into the one or more holes and fuse said sheets to form the construct.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-D are SEM cross-section micrographs of a lyophilized decellularized bovine pericardium (DBP) component.

FIG. 2 shows a high magnification (×200,000) SEM micrograph of the DBP.

FIGS. 3A-C present structures of tissues after applying increasingly high pressures: 100 MPa, 200 MPa and 500 MPa, respectively, far beyond the pressure required to engineer the DBP/polymer construct, which are typically below 1 MPa, or below 0.3 MPa. The fact that the collagen fibrils fully retain their structure after the exceedingly high pressures, demonstrates the robustness of DBP and its remarkable resistance to extremely high pressures.

FIGS. 4A-B show holes made with 25 G and 27 G needles, respectively, filled with a polymer phase, Tecoflex.

FIG. 5 presents coated struts of a metallic stent, when compressed in the left-hand side, and once expanded, in the right-hand side. The weldable polymer used consists of PCL and HDI.

FIG. 6 shows partial welding of a patch to only a small part of coated struts of a metallic stent (around 15% of the area of the stent).

FIG. 7 shows a stent stretched three times its initial length, with no detachment of the patch from the stent occurring.

FIG. 8 shows a structure comprising DBP and the bi-component construct comprising DBP and the polymer phase, in this case Tecoflex. The six holes made in the DBP to enhance the flow of Tecoflex aimed to achieve a strong connection between the two DBP phases and the polymeric phase are readily seen. By doing so, in addition to the diffusion of Tecoflex molecules in the DBP phase, a laminate comprising two external thin Tecoflex films that are continuously connected through the holes made in two DBP phases, to a central Tecoflex film, is formed.

FIGS. 9A-C show DBP leaflets of a heart valve comprising also a metallic frame connected together via polymer connections, e.g., a polyurethane, Tecoflex. In this case, the DBP leaflets are welded together via the polymer phase of the DBP/polymer construct and also to the metallic frame of the heart valve via its coated struts.

FIG. 10 shows a stent-graft used to treat Abdominal Aortic Aneurysms (AAA) among other indications, comprising a metallic stent and a fabric where the latter is sewn together to the former via multiple suturing points.

FIG. 11 demonstrates a polymeric phase consisting of a PCL/HDI poly(ester-urethane) and a low molecular weight polymerizable molecule being hydroxyethyl methacrylate (HEMA). It further reports that CLUR2k has a tensile modulus of around 180 MPa when not plasticized (100:0), and with the addition of increasing amounts of the smart HEMA component, it significantly decreases, showing a value of about 60 MPa in the presence of 20% monomeric HEMA, down to 6 MPa in the 50:50 composition. As apparent from the data presented, polymerized PHEMA results in a significant increase in CLUR's modulus, reaching a value above 250 MPa, for the CLUR2K:PHEMA 50:50 composition.

FIG. 12 presents CLUR2K's behavior when the low molecular component is not only polymerizable but crosslinkable, as in the case of triethyleneglycol dimethacrylate, comprising two carbon double bonds.

FIG. 13 demonstrates a further crosslinking methodology.

FIGS. 14A-B schematically show A) the generation of the tissue/polymer construct, with the first step being the formation of holes in the tissue components, the size, number and array of which is optimized. An embodiment illustrating a “sandwich” of two tissues welded together through and mediator polymer sheet is shown in B).

### DETAILED DESCRIPTION OF EMBODIMENTS

Thus, for the purpose of providing a superior class of biomedical constructs and medical devices, that would lack the deficiencies associated with currently available equivalents, the inventors of the technology disclosed herein have developed a novel and unique class of constructs comprising [i] a decellularized tissue and [ii] a polymeric component, in which each of its components forms a different phase in space, wherein said decellularized tissue and polymeric components are associated with each other via one or more physical anchoring means. Unexpectedly, the constructs and medical devices disclosed hereby display properties previously unattainable.

The decellularized tissue/polymer constructs taught by this invention constitute the whole medical device or are part of it and said constructs include, among others, a vascular graft, a cardiac patch, a stent, a heart valve, a wound or burn dressing, a membrane, a sealing device, or devices that reinforce a suture or staple line, are used in hernia repair, in pelvic floor reconstruction, or in dural closure.

The teachings of the present invention are readily applicable to a diversity of decellularized tissues and polymers. For the sake of clarity, conciseness and simplicity, though, and without detracting from the generality of the scope of the present invention in any form or fashion, the inventors have chosen to illustrate the invention hereby disclosed, by focusing on constructs comprising decellularized pericardium and, more precisely, on decellularized bovine pericardium. For the sake of clarity, conciseness and simplicity, and without detracting from the generality of the scope of the present invention in any form or fashion, the inventors have chosen to illustrate the invention hereby disclosed, by focusing on a medical device where the decellularized pericardium/polymer construct is part of a larger medical device, and more precisely, a heart valve. For the sake of clarity, conciseness and simplicity, though, and without detracting from the generality of the scope of the present invention in any form or fashion, the inventors have chosen to illustrate the invention hereby disclosed, by focusing on polyurethanes as the polymeric phase of said decellularized pericardium/polymer construct and, more precisely, where said polyurethanes is Tecoflex.

Among them and without any limitation, the decellularized bovine pericardium/polymer constructs disclosed by the current invention were developed in the inventor's laboratory. The mechanical properties of the decellularized pericardium somewhat vary with the batch and as a function of the decellularization technique used. Typically, the stress at break of the decellularization technique falls in 10-80 MPa range, its typical Young's modulus values span between 80 and 300 MPa, exhibiting 20-50% strain at break values. The stress at break, Young's modulus and strain at break values measured at the inventors' lab for lyophilized decellularized bovine pericardium were 75 MPa, 280 MPa and 43%, respectively.

Tecoflex polyurethane is an aliphatic polyether urethane comprising a polytetramethylene oxide soft segment and a methylene dicyclohexane diisocyanate (MDI Richards JM, McClennen WH, Meuzelaar HLC, Shockcor JP, Lattimer RP: Determination of the structure and composition of clinically important polyurethanes by mass spectrometric techniques. Journal of Applied Polymer Science 1987, 34:1967-1975).

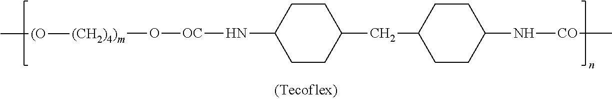

Tecoflex's mechanical properties were measured at the inventors' lab and are shown in the Table 1.

TABLE 1
mechanical properties of Tecoflex

| | Young's | | | |
|---|---|---|---|---|
| Stress at | Modulus | Strain at | Strain at | Stress at |
| yield (MPa) | (MPa) | peak (%) | break (%) | break (MPa) |
| 175 | 50 | 500 | 520 | 169 |

It is apparent from the mechanical data presented that Tecoflex is a very strong material and will not represent the weak component of the constructs. Other polyurethanes such as Elastolan can be used, as well as other polymers displaying similar rheological and mechanical properties. In cases that the construct requires that the polymeric component is biodegradable, polymers such as polylactic acid (PLA), poly(lactic acid/glycolic acid) (PLGA), polycaprolactone (PCL) and their copolymers, as well as more flexible biodegradable polymers such as biodegradable block copolymers, plastic or elastomeric, comprising hydrophilic polyethers such as polyethylene oxide (PEO) or their hydrophobic counterparts, e.g., polypropylene oxide (PPO) or polytetramethylene oxide (PTMO), or flexible aliphatic polyesters such as amorphous polycaprolactones, or silicone-based segments comprising polydimethyl siloxane (PDMS), among numerous others. The molecular weight of the soft segments typically varies from 600 to 20,000 dalton.

For the sake of clarity, conciseness and simplicity, though, and without detracting from the generality of the scope of the present invention in any form or fashion, the inventors have chosen to illustrate the invention hereby disclosed, by focusing on constructs wherein the polymeric phase of the construct is able to connect two or more of the tissue phases or other phases comprising materials of all types selected from a group including polymers, metals, ceramics, carbonaceous materials and combinations thereof. More precisely, the inventors have chosen to illustrate the invention hereby disclosed, by focusing on constructs wherein the polymeric Tecoflex phase of the construct connects two decellularized bovine pericardium tissue samples. Even more specifically, the inventors have chosen to illustrate the invention hereby disclosed, by focusing on constructs that are part of a heart valve comprising also a metallic frame, and connect the leaflets between them and also to the metallic frame of the valve. In some embodiments of the present invention, the tissue/polymer construct is part of decellularized pericardium leaflets. In yet other embodiments of the invention, the polymeric Tecoflex phase of the construct connects two tissue leaflets, whereby a multilayered, integrative tissue/polymer construct is formed comprising three layers of the polymer, two of them external and one in the middle of said construct and two layers of tissue internal to said two external layers of the polymer layers, forming a laminate. In other embodiments, said multilayered, integrative tissue/polymer construct formed consists of polymer layers connected by polymeric connections spanning in dimensions from the nanometric to the centimetric scale.

### Materials and Methods

### Lyophilization of Pericardium

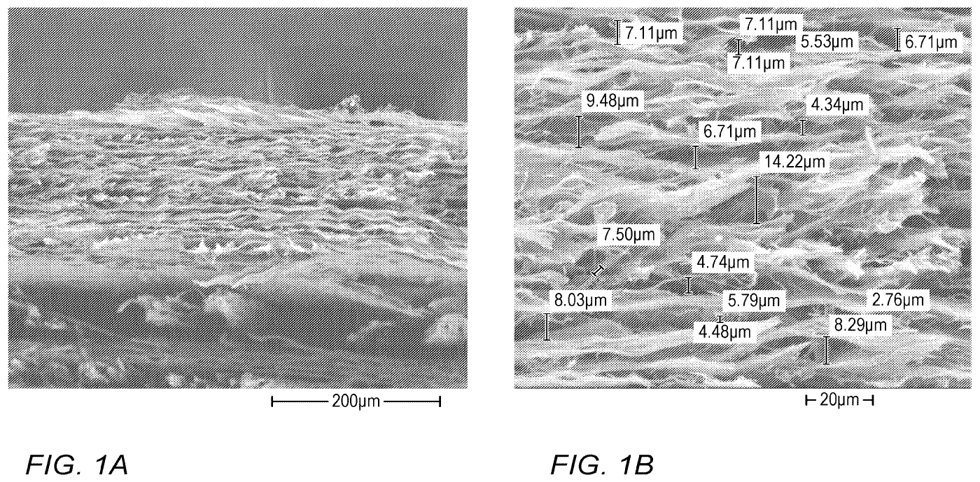

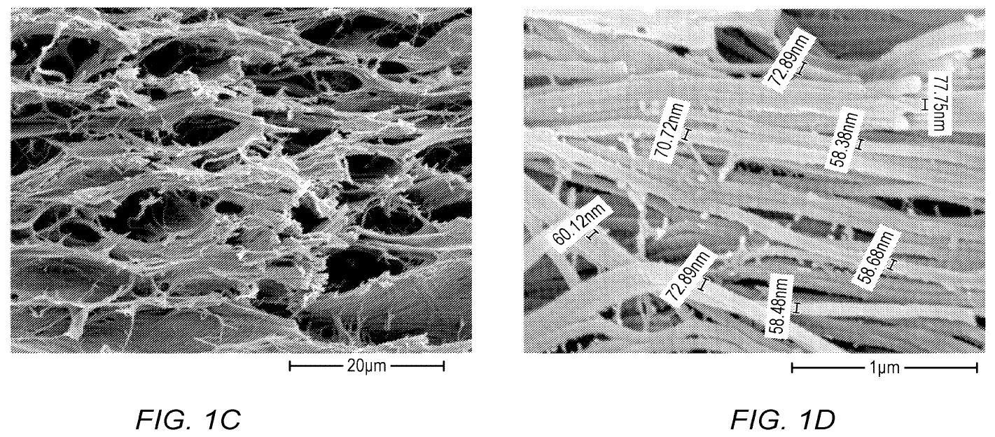

Since in most cases the decellularized tissue component of the construct has to be lyophilized prior to the generation of the construct, the decellularized tissue component was studied following lyophilization. The structure of the lyophilized decellularized bovine pericardium (DBP) component is shown in the SEM cross-section micrographs exhibited in the FIGS. 1A-D below.

The magnification increases from a relatively low ×500 value, with the SEM micrograph showing the whole thickness of the DBP, up to a high ×120,000 value, where the well-known structure of collagen fibrils is readily observed.

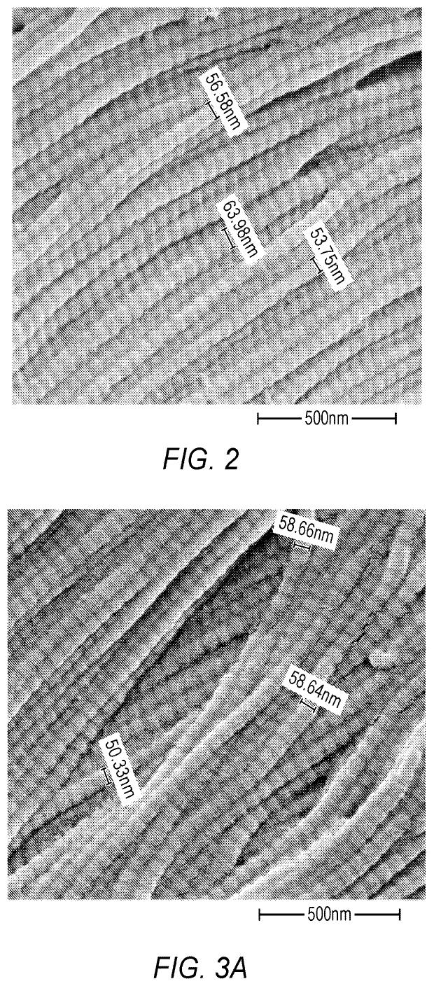

FIG. 2 shows the high magnification (×200,000) SEM micrograph of the as-received DBP.

### Preparation of the Polymer

A selected polymer such as Tecoflex, was added to the welding area in two different states: solution in THE and preprepared film of Tecoflex. The Tecoflex solution was in the range of 5-25% of Tecoflex in THE (refers hereinafter as the “polymer solution”). For the film preparation 3.78 gr of Tecolfex was added to 100 ml of THF until it fully dissolved. This solution was poured into a glass petri dish, and the THF was left to evaporate slowly. Films had an average thickness of several micron.

### Welding of the Pericardium

Two dry pericardium stripes were prepared for the welding process. On each one of them, area of 2 mm from the edge was marked and 6 holes were generated. The holes were introduced with the polymer solution by syringe with a 25 G needle at its tip. The needle was inserted inside the fabric from the rough side to the smooth side. Once it pulled back outside, the solution was poured, simultaneously. The holes were aligned in a straight, parallel to the edge line, in the middle of the welding area.

The pierced fabrics were left for several minutes for further solvent evaporation and then welded at the sealer, with the rough sides pressed towards each other. A sheet of Tecoflex film was inserted in between the pressed fabrics right before the welding process took place.

For the welding process three rounds of sealing were applied in delicate regime which is a combination of temp, pressure, and time of the press with relaxation between each cycle out of three cycles. After the welding was performed, the welded materials were dried at room temperature.

### Welding Characterization Methods

The strength of the welded stripes was analyzed by the Instron instrument by a 10 mm/min tensile testing, whereas the leaflets were gripped 10 mm from the welded area at both its sides.

Also, the structure of the welded stripes was tested by SEM to ensure that the structure had no major changes because of the press and the heat.

The results are provided in Tables 2-4 below:

TABLE 2
Tensile test results for a Tissue to
Tissue welded model with no holes

| | | | | Stress @ |
|---|---|---|---|---|
| | Force @ | Width | Thickness | Peak |
| Test No | Peak (N) | (mm) | (mm) | (MPa) |
| 1 | 0.447 | 6.0 | 0.4 | 0.186 |
| 2 | 0.914 | 6.0 | 0.4 | 0.381 |
| 3 | 0.426 | 6.0 | 0.4 | 0.178 |
| Min | 0.426 | 6.0 | 0.4 | 1.178 |
| Mean | 0.596 | 6.0 | 0.4 | 0.248 |
| Max | 0.914 | 6.0 | 0.4 | 0.381 |
| S.D. | 0.276 | | 0.0 | 0.115 |

TABLE 3
Tensile test results for a Tissue
to Tissue welded model with 4 holes

| | | | | Stress @ |
|---|---|---|---|---|
| | Force @ | Width | Thickness | Peak |
| Test No | Peak (N) | (mm) | (mm) | (MPa) |
| 1 | 9.084 | 6.0 | 0.4 | 3.785 |
| 2 | 7.38 | 6.0 | 0.4 | 3.075 |
| 3 | 9.017 | 6.0 | 0.4 | 3.757 |
| Min | 7.38 | 6.0 | 0.4 | 3.075 |
| Mean | 8.494 | 6.0 | 0.4 | 3.539 |
| Max | 9.084 | 6.0 | 0.4 | 3.785 |
| S.D. | 0.965 | | 0.0 | 0.402 |

TABLE 4
Tensile test results for a Tissue
to Tissue welded model with 6 holes

| | | | | Stress @ |
|---|---|---|---|---|
| | Force @ | Width | Thickness | Peak |
| Test No | Peak (N) | (mm) | (mm) | (MPa) |
| 1 | 9.057 | 6.0 | 0.4 | 3.6228 |
| 2 | 16.156 | 6.0 | 0.4 | 6.4624 |
| 3 | 8.586 | 6.0 | 0.4 | 3.4344 |
| Min | 8.586 | 6.0 | 0.4 | 5.248 |
| Mean | 11.266 | 6.0 | 0.4 | 4.507 |
| Max | 16.156 | 6.0 | 0.4 | 13.463 |
| S.D. | 4.241 | | 0.0 | 1.696 |

The data presented in Tables 2-4 support the understanding that the more holes filled with the polymer are formed on the tissues, the stronger the association or welding is.

### Discussion

Applying mild pressure may be necessary in some embodiments of the invention, especially when the polymeric phase connects more than one DBP and/or polymeric phases. The objective of applying pressure in these embodiments of the invention aims at causing the polymer phase or phases to flow, in some embodiment in contact with the DBP and in others when in contact with other polymeric phases, and combinations thereof.

In some embodiments of this invention, the polymeric phase can flow through pores or holes made in the tissue phase, and polymer molecules can also diffuse and intermingle with molecules of other phases. In some embodiments, the flow of the polymeric molecule causes the interface between the phases to vanish, welding them together.

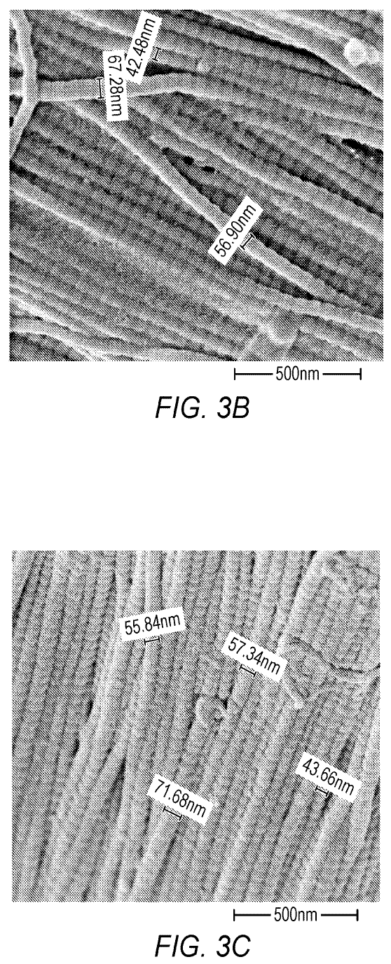

FIGS. 3A-C present the structure of the tissue after applying increasingly high pressures, 100 MPa, 200 MPa and 500 MPa, respectively, far beyond the pressure actually required to engineer the DBP/polymer construct, which are typically below 1 MPa, preferably below 0.5 MPa and even more preferably below 0.3 MPa. The fact that the collagen fibrils fully retained their structure after these exceedingly high pressures, demonstrates the robustness of DBP and its remarkable resistance to extremely high pressures.

Several polymers that are able to form the polymer phase of the DBP/polymer construct have demonstrated their ability to flow under physiologically acceptable pressure and temperature conditions. Thermograms of two biodegradable poly(ester-urethane)s consisting of polycaprolactone (PCL) of different molecular weights (2,000 and 14,000 dalton) and hexamethylene diisocyanate (HDI), and of their welded phase, obtained by Differential Scanning Calorimetry (DSC), demonstrated new, broader endotherms, shifted to lower temperatures. This provides an indication that the molecules of both polymers have inter-diffused, one hampering the crystallizability of the other.

This phenomenon and the strength of the welding connection were further explored where two films of two different polyurethanes, Tecoflex and PEU, were welded together. The results demonstrate the ability of Tecoflex and PEU chains to flow across the interface between the two films, providing a strong connection of long term clinical importance. Additionally, both polymers are similar since they are polyurethanes, even though in the first case they are poly(ester-urethane)s and in the latter case they are poly(ether-urethane)s. To illustrate the broad scope of this phenomenon, the DSC thermograms of two polymers were explored.

DSC demonstrated that the two polymers were very different, one being flexible poly(ester-urethane) and the other a stiff polymethacrylate. The flexible polyurethane consists of PCL 2000 segments and HDI as their coupling agent, while the rigid polymethacrylate is poly(ethylmethacrylate) (PEMA). Also in this case, even though the polymers substantially differ in their composition and mechanical properties, it is apparent from the thermogram of their welded phase, that the phenomenon entails intermingling and entanglement of the chains of both components at the molecular level. In several embodiments of this invention, the mobility of the polymer chains and their ability to disentangle, cross an interface with another polymeric or decellularized tissue phase, diffuse into the second phase and then re-entangle, plays a key role in generating the constructs or medical devices taught by this invention.

The strength of the connection between the DBP and polymer phases, e.g., Tecoflex, was quantitatively determined using the Instron machine. It was found that the construct failed cohesively, within the tissue, and no de-welding failures were observed.

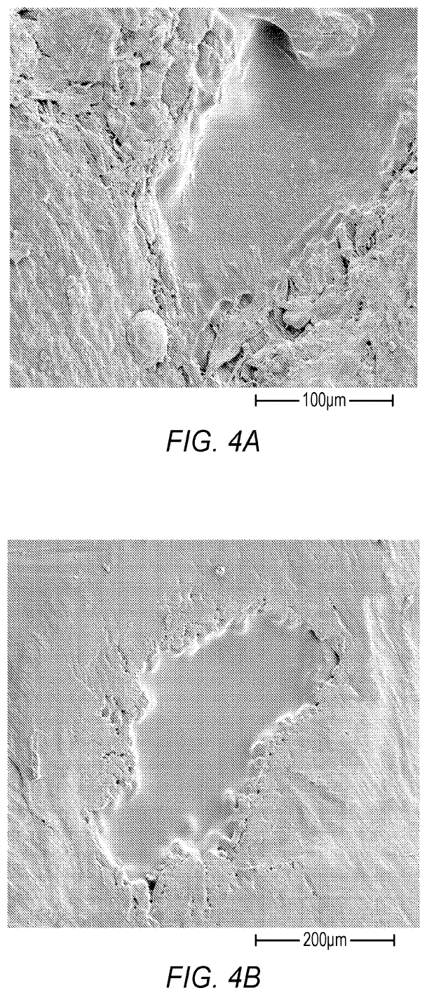

In some embodiments, where an especially strong connection between the DBO phase and the polymer phase was aimed at, holes were made in the DBP phase, to maximize the flow of the polymer phase through them. In some embodiments the holes were made using needles in the 16 G to 27 G range. FIGS. 4A-B show the holes made with 25 G and 27 G needles, respectively, filled with a polymer phase, Tecoflex in these cases.

The DBP phase and the polymer phase can be of any size, spanning from nanometric to centimetric and can adopt any shape, including, without limitation, spherical, fibrous, strips, ribbons, a film, porous or not, and combinations thereof. The polymeric phase/s can also be present in phases the size of which range from being nanosized up to being in the centimeters scale, and they can be on the surface of a DBP phase and/or in its bulk, and each of these cases being of a size ranging from nanometers to centimeters and adopting any geometry.

It is an objective of this invention to connect the DBP/polymer construct or the device the construct is part of to an additional component of the device. In the case of heart valve, a much-preferred embodiment of this invention, said additional component is the metallic frame of the heart valve.

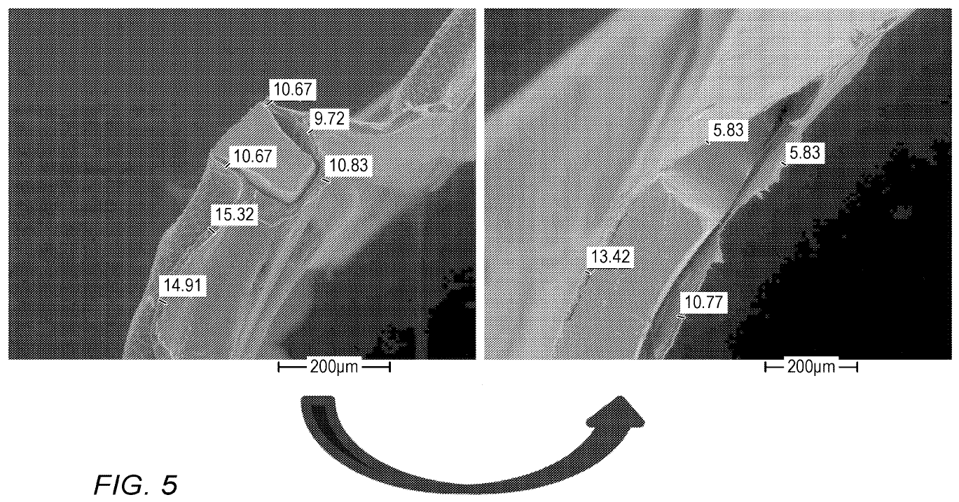

In some embodiments of the present invention, when welding the DBP/polymer construct or the device the construct is part of to a metallic stent, the struts of the stent are coated with a weldable polymer that can be the same or different from the polymer constituting the polymer phase of the DBP/polymer construct. FIG. 5 presents the coated struts of a metallic stent, when compressed in the left hand side, and once expanded, in the right hand side. In this embodiment, due to the large expansion of the stent, the weldable polymer coating its struts was chosen to be especially flexible and displaying a high strain at break value. In the case shown below, the weldable polymer used consisted of PCL and HDI. Coatings of different thicknesses were prepared, starting with coating as thin as 5 micrometers and increasing as required.

Other highly flexible polymers were used as well. Among others, various polyurethanes were used, their soft segment consisting of polyethers or polyesters varying in their molecular weight, their hydrophilicity and, in the case of biodegradable polymers, also in their rate of degradation. One of the polymers used consists of poly(tetramethylene oxide) (PTMO) (MW=650) segments chain extended via HDI. As reported in Table 5, the polymer was welded within 20 seconds at a 47-48° C. temperature.

TABLE 5
Polymer properties

| | P(PTMG650) |
|---|---|
| Patch material | P(PTMG650) |
| T at balloon surface | 47-48° C. |
| Welding time | ~20 s |

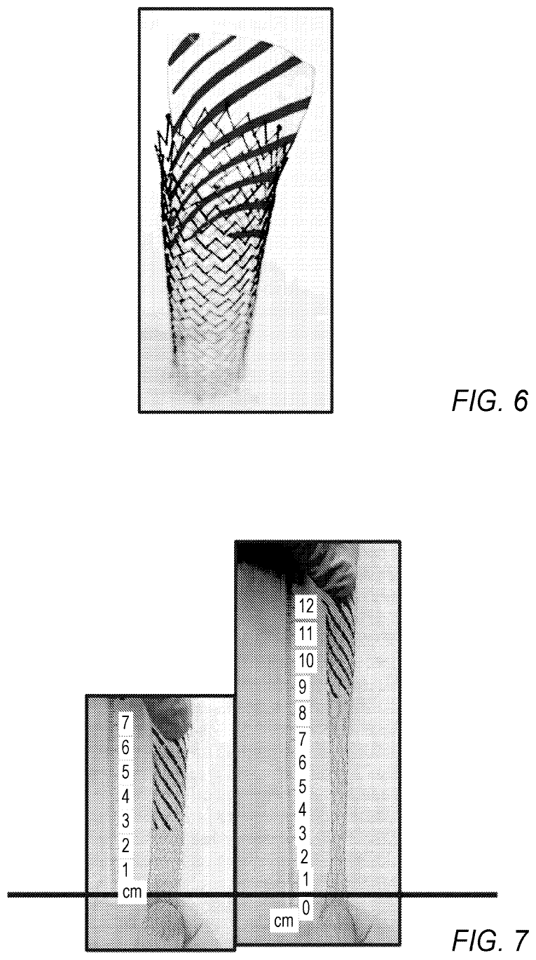

The same polymer was used to coat the struts of the stent s also to create the polymer phase of the DBP/polymer construct and of the additional polymer phase of the medical device, the polymer is part of. To illustrate not only the speed of the welding process but also the strength of the polymer/polymer connection formed, a patch of the PTMO650/HDI polymer was welded in 20 seconds to the struts of the stent coated with the same polymer. Furthermore, the patch was only allowed to weld to only a small part of the coated struts of the metallic stent, as shown in FIG. 6 (around 15% of the area of the stent).

Initially, forceful manual efforts were made to de-weld the patch from the coated stent but, as shown in FIG. 7, the stent was stretched three times its initial length, with no detachment of the patch from the stent occurring.

When finally the Instron instrument was used to de-weld the patch from the stent, unexpectedly and surprisingly, it was the metallic stent that failed, while the welded connection remained unaffected. This compelling demonstrates the advantageous features that stem from the ability of the polymer phase of the DBP/polymer construct or of the polymer phase of the medical device the construct is part of to, rapidly and strongly, form long-term, stable connections between the different phases.

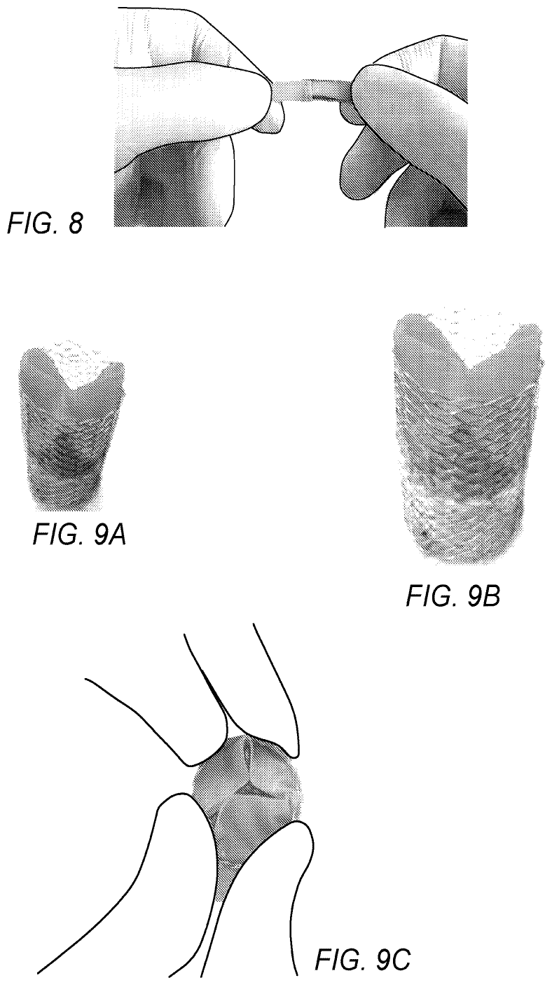

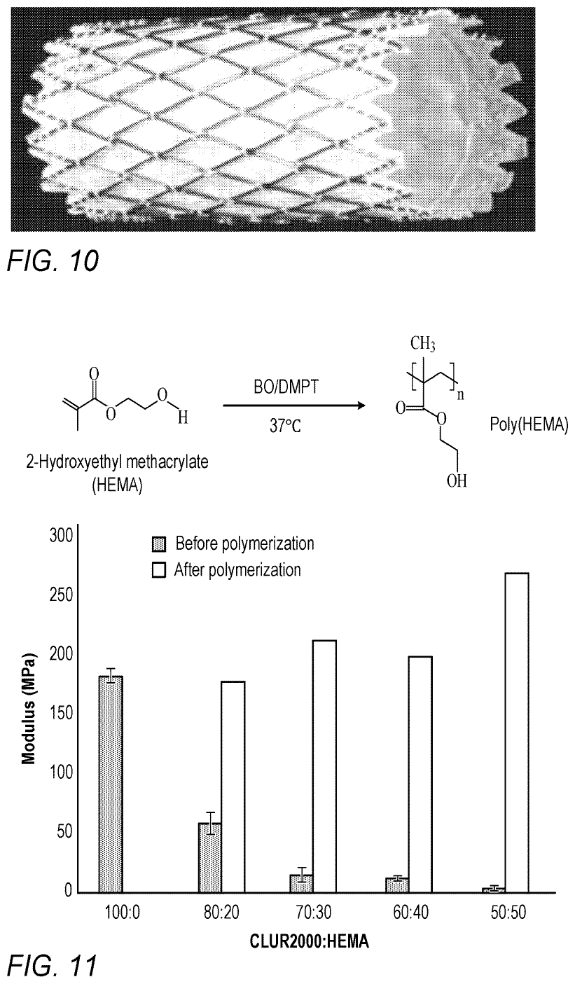

FIG. 8 shows a structure comprising DBP and the bi-coponent construct comprising DBP and the polymer phase, in this case Tecoflex. The six holes made in the DBP to enhance the flow of Tecoflex aimed to achieve a strong connection between the two DBP phases and the polymeric phase are readily seen. By doing so, in addition to the diffusion of Tecoflex molecules in the DBP phase, a laminate comprising two external thin Tecoflex films, that are continuously connected through the holes made in two DBP phases, to a central Tecoflex film, is formed.

In a much-preferred embodiment, DCP leaflets of a heart valve comprising also a metallic frame are connected together via polymer connections, preferably a polyurethane, more preferably Tecoflex, as shown in FIGS. 9A-C. In this case, the DBP leaflets are welded together via the polymer phase of the DBP/polymer construct and also to the metallic frame of the heart valve via its coated struts.

As usually done and routinely reported in the literature, the long-term dynamic stability of heart valves is determined in vitro by the Accelerated Wear Tester (AWT), under accelerated conditions.

References to three articles, representative of the many studies that describe the use of AWT follow in heart valves follow: {1} A correlation between long-term in vitro dynamic calcification and abnormal flow patterns past bioprosthetic heart valves, Oleksandr Barannyk, Robert Fraser and Peter Oshkai, J Biol Phys (2017) 43:279-296); {2} Pitfalls and outcomes from accelerated wear testing of mechanical heart valves, A Campbell, T Baldwin, G Peterson, J Bryant and K Ryder, J Heart Valve Dis, 1996 June; 5 Suppl 1:S124-32; discussion 144-8; {3}. A study in the design of an Accelerated Wear Tester that is compatible with a particle image velocimetry and high-speed camera setup, Edward A. Brown, Master of Science thesis, The Pennsylvania State University, The Graduate School, College of Engineering, 2015.).

During these tests, a maximum cyclic stress of 100 mmHg is applied, which equals to 0.0133 MPa (12.3 kPa). The failure values measured using the Instron instrument typically fell in the 5 to 10 MPa range, more than 300 higher than the maximum cyclic stress applied during the AWT determinations.

The advantageous features of the DBP/polymer constructs taught by this invention, become even more striking and surprising, when compared to the routinely used alternative suturing technique.

Connecting the phases of the construct and the construct to other phases of the medical device the construct is part of have important advantages over suturing: [a] It is very fast as opposed to the extremely tedious and time consuming suturing process, via numerous suturing points, [b] generates strong connections, [c] is reproducible and not technician dependent, [d] achieves much better compliance match between the phases of the construct and the device, in striking contrast to the much stiffer sutures used, [e] achieves a much more homogeneous distribution of stresses, as opposed to the extremely detrimental and sometimes life threatening stress concentrating effect of sutures, and [f] is inexpensive.

In some embodiments, any of the phases of the construct and/or the medical device the construct is part of or any element of the invention may comprise at least one additional material to improve any aspect of the clinical performance of any of the embodiments of the invention and combinations thereof, including its biocompatibility, its hemocompatibility, the cellular response they trigger, among others. The at least one additional material may be selected amongst active and non-active materials. In some embodiments, the active materials are selected from a variety of bioactive agents. Exemplary bioactive agents include, for example, anticoagulants, such as heparin and chondroitin sulphate; fibrinolytics such as tPA, plasmin, streptokinase, urokinase and elastase; steroidal and non-steroidal anti-inflammatory agents such as hydrocortisone, dexamethasone, prednisolone, methylprednisolone, promethazine, aspirin, ibuprofen, indomethacin, ketoralac, meclofenamate, tolmetin; calcium channel blockers such as diltiazem, nifedipine, verapamil; antioxidants such as ascorbic acid, carotenes and alpha-tocopherol, allopurinol, trimetazidine; antibiotics, such as noxythiolin and other antibiotics to prevent infection; prokinetic agents to promote bowel motility; agents to prevent collagen crosslinking such as cis-hydroxyproline and D-penicillamine; and agents which prevent mast cell degranulation such as disodium chromolglycate, among numerous others.

In addition to the above agents, which generally exhibit favorable pharmacological activity related to promoting wound healing or reducing infection or having hemostatic properties or enhancing hemocompatibility, other bioactive agents may be delivered by the constructs or the medical devices of the present invention that include, for example, amino acids, peptides, proteins, including enzymes, carbohydrates, growth factors, antibiotics (treat a specific microbial infection), anti-cancer agents, neurotransmitters, hormones, immunological agents including antibodies, nucleic acids including antisense agents, fertility drugs, psychoactive drugs and local anesthetics, among numerous additional agents. The delivery of these agents and others will depend upon the pharmacological activity of the agent, the site of activity within the body and the physicochemical characteristics of the agent to be delivered, the therapeutic index of the agent, among other factors. One of ordinary skill in the art will be able to readily adjust the physicochemical characteristics of the present polymers and the hydrophobicity/hydrophilicity of the agent to be delivered in order to produce the intended effect. In this aspect of the invention, bioactive agents are administered in concentrations or amounts which are effective to produce an intended result. It is noted that the chemistry of polymeric phases according to the present invention can be modified to accommodate a broad range of hydrophilic and hydrophobic bioactive agents and their delivery to sites in the patient.

In some embodiments, the non-active materials are selected amongst dyes, polymeric materials, thickening agents, plastizicers, agents affecting hydrophilicity, agents affecting lubricity and others.

The constructs and medical devices taught by the invention may be manufactured by any of the existing manufacturing techniques, such as extrusion, compression molding, injection molding, dip coating, solvent casting, welding, any of the numerous 3D printing techniques, and in each case the specific manufacturing technique being used will be tailored so it is compatible with the constructs and medical devices taught by the invention.

In some embodiments where at least one of the polymer phases is biodegradable, said biodegradable polymer is selected from a group comprising lactic acid, lactide, glycolic acid, glycolide, or a related aliphatic hydroxycarboxylic acid or ester (lactone) selected from the group consisting of. β-propiolactone, ε-caprolactone, δ-glutarolactone, δ-valerolactone, β-butyrolactone, pivalolactone, α,α-diethylpropiolactone, ethylene carbonate, trimethylene carbonate, γ-butyrolactone, p-dioxanone, 1,4-dioxepan-2-one, 3-methyl-1,4-dioxane-2,5-dione, 3,3,-dimethyl-1-4-dioxane-2,5-dione, cyclic esters of α-hydroxybutyric acid, α-hydroxyvaleric acid, α-hydroxyisovaleric acid, α-hydroxycaproic acid, α-hydroxy-α-ethylbutyric acid, α-hydroxyisocaproic acid, α-hydroxy-α-methyl valeric acid, α-hydroxyheptanoic acid, α-hydroxystearic acid, α-hydroxylignoceric acid, salicylic acid and mixtures, thereof.

The polymeric phases according to the present invention comprise optionally low molecular weight molecules able to enhance the flowability of said polymeric and/or allow causing the polymer phase or part of it to flow under milder temperature and pressure conditions. It is a further object of the invention to provide low molecular weight molecules that are polymerizable or crosslinkable, so they soften the polymer phase or parts of it before they polymerize or crosslink and strengthen or stiffen said polymer phase once polymerized or crosslinked, where said low molecular weight molecules can polymerize or crosslink following any mechanism including, without limitation, addition and condensation polymerization reactions as well as additional reactions, including all types of click chemistry and combinations thereof. Among others, said polymerizable or crosslinkable low molecular weight molecules include precursors comprising one or more double bonds. A few examples are given in the figures below.

In FIG. 11, the polymeric phase consists of a PCL/HDI poly(ester-urethane) and the low molecular weight polymerizable molecule is hydroxyethyl methacrylate (HEMA).

FIG. 11 reports that CLUR2k has a tensile modulus of around 180 MPa when not plasticized (100:0), and with the addition of increasing amounts of the smart HEMA component, it significantly decreases, showing a value of about 60 MPa in the presence of 20% monomeric HEMA, down to 6 MPa in the 50:50 composition. As apparent from the data presented, polymerized PHEMA results in a significant increase in CLUR's modulus, reaching a value above 250 MPa, for the CLUR2K:PHEMA 50:50 composition.

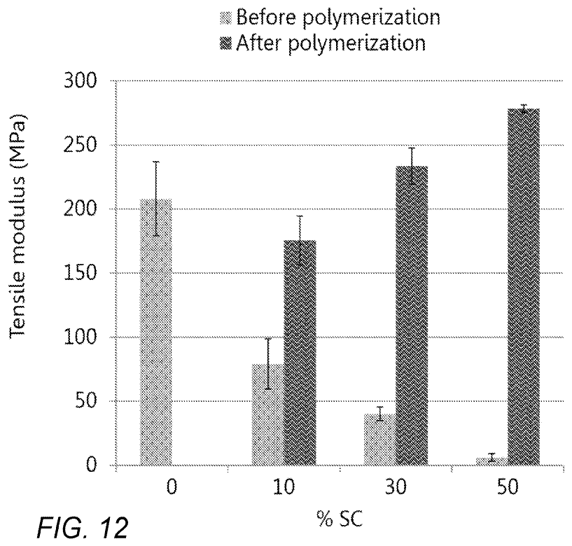

FIG. 12 presents CLUR2K's behavior when the low molecular component is not only polymerizable but crosslinkable, as in the case of triethyleneglycol dimethacrylate, comprising two carbon double bonds.

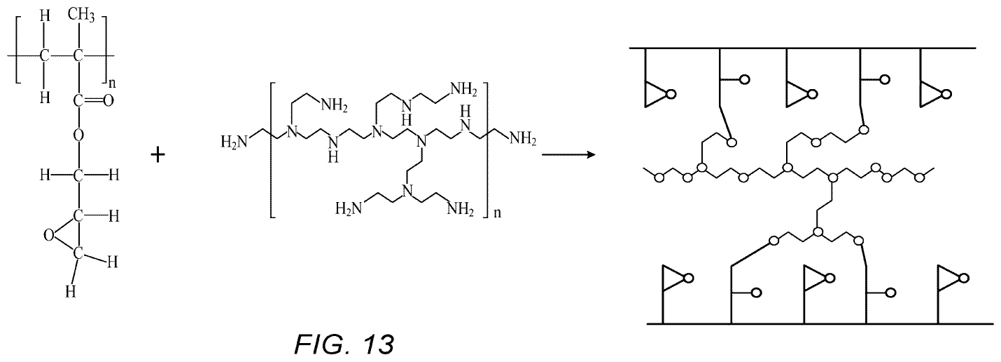

Several additional chemistries can be employed to polymerize or cross-link the low molecular weight component, such as the epoxy-amine reaction. This chemistry is illustrated by using polyglycidyl methacrylate (PGMA) (see FIG. 13), is very stiff and somewhat brittle, blended with polyethylene imine molecules, which contain several amine groups. When initially blended with PGMA, PEI molecules lower PGMA's rigidity but, once it reacts with the epoxide ring (see FIG. 13), it crosslinks PGMA and stiffens the polymer.

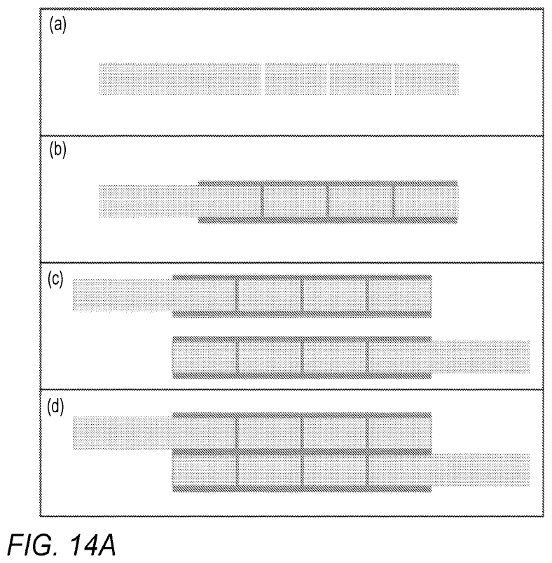

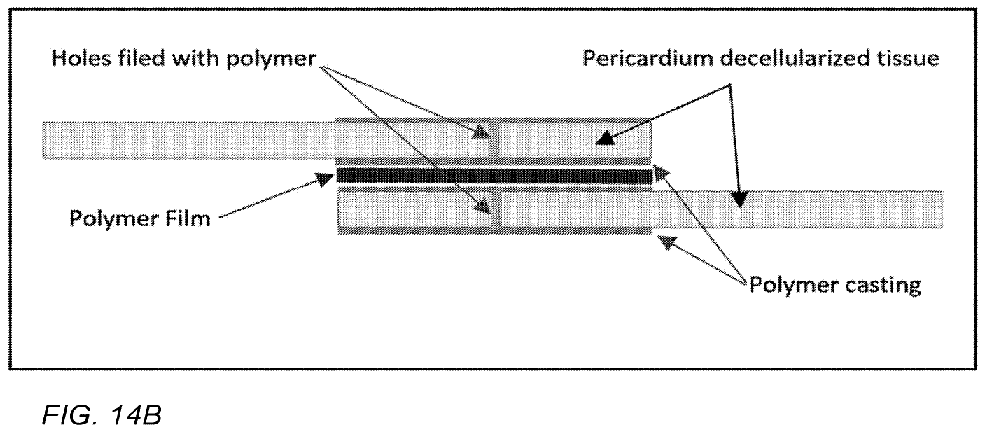

FIG. 14 schematically shows the generation of the tissue/polymer construct, with the first step being the formation of holes in the tissue components, the size, number and array of which is optimized. In this case a liquid polymer phase is added, which may be for example a polymer solution in an appropriate solvent, or when the polymer is above its glass transition or melting point, when these transitions occur at a suitably low temperature. The polymer penetrates the tissue, primarily through the holes made in it, the length, size, number and array of which is controlled and varies over a significant range. In some instances, the polymer phase contains holes of different depth of penetration. In some instances, holes penetrate the tissue phase only partially and in other embodiments, the depth of the holes cross face-to-face the tissue phase. In the case depicted below, the holes cross the whole tissue thickness, generating on both side of the tissue component, two layers of polymer. Since they are connected via the polymer connections filling the holes, the two polymer films become one integrative polymer phase. In this case, the mechanical properties of the construct created by the physical association of the tissue and polymer phases are especially high, since they derive from the cohesive strength of the polymer itself.

In other embodiments of the invention, a pre-formed polymer phase is first produced, the composition and morphology of which are such that it can flow under the right temperature and pressure conditions, as described before.

In some embodiments, said pre-formed polymer phase has different properties in its surface layers, as opposed to its bulk, which allow the polymer phase in contact with the tissue to flow into the holes made in the tissue. In some embodiments, the difference between the surface layers, the thickness of which is optimized, and the bulk of the polymer phase, is compositional, so that the surface layers exhibit the required flowability under the temperature and pressure conditions applied. In some embodiments, the difference between the surface layers, the thickness of which is optimized, and the bulk of the polymer phase, is morphological. In this embodiment, the surface layer is less crystalline, and in some embodiments, amorphous, while the polymer in the bulk of the polymer phase displays enhanced crystallinity and, therefore, higher rigidity at the relevant temperature. The morphological differential encoded in the polymer phase can be achieved following various strategies. Among other, this can be achieved conducting spatially confined thermal treatments that render the surface layer less crystalline or amorphous, as opposed to the bulk of the polymer phase. In some embodiments, the difference between the surface layers and the bulk of the polymer phase is achieved by the addition of a mobile component that tend to migrate and concentrate on the surface layers of the polymer phase. In yet other embodiments, the difference between the surface layers and the bulk of the polymer phase is achieved by judiciously chosen the surfaces in contact with which the polymer phase is produced. These and other similarly effective techniques can be used separately or can be combined.

In yet additional embodiments of the invention, an initially liquid polymer phase and a pre-formed polymer phase are combined in different ways. In some embodiments, for example, they are added simultaneously, or they can also be deployed sequentially, or in any other manner that will produce a tissue/polymer optimal association, as derived from its clinical use.

The procedure whereby a tissue/polymer consisting of at least two tissue phases is produced using a polymer solution, is exemplified hereby and depicted in FIG. 14B. In some instances, the term “welding” is used interchangeably with “connecting” and similar terms. Two lyophilized pericardium strips were used and the tissue/polymer association resulted in the connection between two tissue phases via the polymer phase. On each one of the pericardium strips, 6 holes were generated within an area of 2 mm from the edge, spaced throughout the width of the tissue strip. In this embodiment, the holes were formed and filled with a polymer solution simultaneously. In this case, an 8% Tecoflex/THF solution was prepared, and a syringe with a 25 G needle at its tip was filled with the solution (for other purposes on in other experiments, a concentration of 1% or 4% Tecoflex was used). The needle was inserted in the pericardial tissue strip from its rough side puncturing it and creating the holes, and the polymer solution was poured initially distally and then while pulling back, filling the holes, and finally generating a polymer film on the tissue proximal face. By doing so, a laminate consisting of two external polymer phases connected via polymer connections (through the holes made in the tissue), was produced. The holes were aligned in a straight, parallel to the edge line, in the middle of the welding area. The tissue/polymer constructs consisting of the laminate described above were left for 2 minutes to allow optimal solvent (THF in this specific case) evaporation and then the two treated tissue/polymer constructs of each tissue strips were overlapped as required and welded together under the right temperature, pressure and time conditions, using a sealer, with the rough sides pressed towards each other. Three rounds at the sealer were applied at 85° C. Each round at the sealer consisted of a 2 bar, 12 seconds step, followed by 1 second of relaxation between the cycles. After the connection was performed, the welded tissue/polymer constructs were dried at room temperature for 5 hours, followed by an immersion in saline solution for 24 hours. The strength of the connection between the two connected tissue strips was studied at the Instron instrument using a 10 mm/min cross-head speed, with the strips being gripped 10 mm away from the connected area.

The strength of the connection formed was also determined under peel conditions and to this end, samples were connected accordingly. All other details of the procedure remained the same. The mechanical studies of these samples were conducted using the same conditions as previously indicated, with a sole difference—the grasp took place 20 mm from the welded area.

Some of the different conditions and connection parameters studied are listed below:
1) For 27 G needle—2/4/6/8 holes.
   For 25 G needle—2/4/6 holes.
   For 23 G needle—2/4/6 holes.
   For 21 G needle—2/4 holes.
   For 19 G needle—2/4 holes.
   For 16 G needle—2 holes.
2) Cycles performed at the sealer—from 1 to 6.
3) Connection pressure—from 2 bar to 10 bar.
4) Connection time—from 8 seconds to 12 seconds.

The procedure whereby a tissue/polymer consisting of at least two tissue phases is produced by combining a polymer solution and a pre-formed polymer phase, is exemplified hereby. The steps involving the Tecoflex/THF solution were the same as described above.

The tissue/polymer constructs consisting of the laminate formed, as described above, were left for 2 minutes to allow optimal solvent (THF in this specific case) evaporation. Before welding the two tissue/polymer constructs prepared, a 2*6 mm Tecoflex film (thickness˜160 μm) was deployed between the two tissue/polymer constructs before the welding process using the sealer, took place. The Tecoflex film was pre-prepared using the solvent casting method using 3.78 g of Tecoflex in 100 ml of chloroform, even though other solvents were also used. The same steps described already were conducted at the sealer and the connected samples were studied at the Instron machine, as described above.

Some of the different conditions and connection parameters studied are listed below:
1) For 27 G needle—2/4/6/8 holes.
   For 25 G needle—2/4/6 holes.
   For 23 G needle—2/4/6 holes.
   For 21 G needle—2/4 holes.
   For 19 G needle—2/4 holes.
   For 16 G needle—2 holes.
2) The pre-generated film was prepared either from THE or Chloroform solvents using 1.5-4 g of Tecoflex in 100 ml of the mentioned solvents.
3) Cycles performed at the sealer—from 1 to 6.
4) Welding pressure—from 2 bar to 10 bar.
5) Welding time—from 8 seconds to 12 seconds.

1-52. (canceled)

53. A construct comprising at least one decellularized tissue and at least one polymeric component, wherein the polymeric component at least partially penetrates at least one surface region of the decellularized tissue.

54. A decellularized tissue physically associated to a polymer component, said association comprising or consisting at least partial penetration of the polymer component into a surface region of the tissue.

55. A construct comprising at least one decellularized tissue and at least one polymeric component, wherein the polymeric component having at least one surface feature protruding one face of the decellularized tissue, crossing it to the other face through at least one hole formed in the tissue.

56. The construct according to claim 53, wherein said at least one decellularized tissue and at least one polymeric component are thermally bonded to provide said construct.

57. The construct according to claim 56, wherein said thermal bonding is achieved by raising the temperature of the polymeric component above either (a) the transition temperature thereof; or, (b) the melting temperature thereof for semi-crystalline polymers; to result, at least partially, in a polymer chain entanglement along at least one region of the polymeric component.

58. The construct according to claim 53, being in a form of a multisheet construct.

59. The construct according to claim 58, wherein the multisheet construct comprising at least one sheet of a decellularized tissue and at least one sheet or segment of a polymeric component, wherein any sheet of the decellularized tissue is adjacent to or in contact with at least one sheet or segment of the polymeric component; and wherein at least two sheets or segments of the polymeric component are associated to each other via at least one hole formed in the at least one sheet of the decellularized tissue.

60. The construct according to claim 58, wherein at least one or any of the at least one sheets of a decellularized tissue is/are confined between any two sheets of the polymeric component.

61. The construct according to claim 58, wherein the multisheet construct comprises a number of sheets of the decellularized tissue and same number of sheets of the polymeric component.

62. The construct according to claim 53, comprising a two or more assemblies of a decellularized tissue confined between two sheets or segments of a polymer component, wherein said assemblies are associated to each other.

63. The construct according to claim 60, wherein at least two assemblies are oppositely oriented.

64. The construct according to claim 53, provided with a wire element or metal frame.

65. The construct according to claim 64, wherein the metal frame is connected to said construct by at least partial coating said metal frame with the polymeric component.

66. The construct according to claim 64, wherein the polymeric component is in a form of a layer or a coat of particles, a polymeric sheet, a polymeric film, a polymeric fiber or a polymeric mesh, a gel, a hydrogel or as a liquid or fluidic film and any combination thereof.

67. The construct according to claim 53, wherein the at least one hole is pre-formed or is present in the decellularized tissue.

68. The construct according to claim 53, wherein the decellularized tissue is selected from pericardium, bovine pericardium, swine pericardium, omentum or small intestine mucosa.

69. The construct according to claim 53, wherein at least one of the following is held true (a) the polymer component is or comprises a polymer selected amongst hydrophobic, hydrophilic, and amphiphilic polymers; (b) the polymer component is or comprises a blend, an IPN, or a semi-IPN; (c) the polymer component is or comprises an acrylic or a methacrylic polymer; (d) the polymer component is or comprises a polyolefin; (e) the polymer component is or comprises a silicone polymer; (f) the polymer component is or comprises a polycarbonate, a polyurethane, a polyurea or a polyamide and combinations thereof, (g) the polymer component is or comprises a polyurethane; (h) the polymer component is or comprises a polymer selected from polymethyl methacrylate (PMMA), poly(n-butyl methacrylate) (PBMA), poly(hexyl methacrylate) (PHMA), polystyrene (PST), poly(2-hydroxyethyl methacrylate) (PHEMA), poly(N-(2-hydroxypropyl)methacrylamide) (PHPMA), polycyanoacrylate (PCA), a polyethylene/polypropylene copolymer, a polyethylene/polybutylene copolymer, a polypropylene/polybutylene copolymer, poly-isobulyene, polydimethylsiloxane (PDMS), phenyl-containing PDMS, polyester urethanes, polyether urethanes, polycarbonate, silicone-containing polyurethanes, polyglycolic acid, polylactic acid, polycaprolactone, polylactide-caprolactone copolymer, polyglycolic acid-lactic acid copolymer, polyethylene oxide-polylactic acid copolymer, polyethylene oxide-polycaprolactone copolymer, polytetramethylene oxide-caprolactone copolymer, polyhydroxy butyrate, polyhydroxy valerate, polyethylene adipate, polybutylene adipate, polyethylene succinate polybutylene succinate and polybutylene terephthalate and polyethylene/butylene terephthalate copolymers and combinations and copolymers thereof; (i) the polymer component is or comprises a shape memory element; (j) the polymer component is or comprises a polyether urethane selected from Pellethane, Elastane, Elastolan, Tecoflex, Biomer; (k) the polymer component is or comprises a polycarbonate urethane selected from Chronoflex, Biospan and Bionate; (1) the polymer component is or comprises a silicone-containing polyurethane selected from CarboSil, PurSil, Avcothane and Cardiothane; (m) the polymer component is or comprises Chronoflex or Tecoflex and any combination thereof (n) any combination thereof.

70. The construct according to claim 53, wherein one or more of the sheets is designed as a material reservoir for releasing active or non-active materials.

71. The construct according to claim 70, wherein the active material is selected from analgesics; antianxiety drugs; antiarrhythmics; antibacterial agents; antibiotics; anticoagulants and thrombolytics; anticonvulsants; antidepressants; antidiarrheals; antiemetics; antifungals; antihistamines; antihypertensives; anti-inflammatories; antineoplastics; antipsychotics; antipyretics; antivirals; beta-blockers; corticosteroids; cytotoxics; hormones and sex hormones; enzymes; and vitamins.

72. A device comprising a construct according to claim 53.

73. The device according to claim 72, configured as an implant.

74. The device according to claim 72, the device is selected from stents, metallic stents, vascular grafts, heart valves, membranes, sealing devices, suture or staple lines, hernia meshes or hernia repair devices, pelvic floor reconstruction devices, wound or burn dressings, dural closures and cardiac patches.

75. A process for manufacturing a construct according to claim 53, the process comprising
contacting at least one pierced surface region of at least one decellularized tissue with at least one polymer, and
permitting said at least one polymer to penetrate into the piercings (holes).

76. The process according to claim 75, further comprising at least one step selected from (a) piercing or forming holes in a surface region of at least one decellularized tissue; (b) inserting the polymer into the piercings (holes); (c) curing the polymer; (d) permitting said at least one polymer to penetrate into the one or more holes to thereby forming a polymeric sheet on the surface region; (e) associating or fusing two or more constructs; (f) thermal bonding of said at least one decellularized tissue and said at least one polymer by raising the temperature of the polymeric component above either (i) the transition temperature thereof; or, (ii) the melting temperature thereof for semi-crystalline polymers to result, at least partially, in a polymer chain entanglement along at least one region of the polymeric component; (g) selecting the polymeric to be in a form of a layer or a coat of particles, a polymeric sheet, a polymeric film, a polymeric fiber or a polymeric mesh, a gel, a hydrogel or as a liquid or fluidic film and any combination thereof; (h) any combination thereof.

77. A process for manufacturing a construct comprising at least one decellularized tissue and at least one polymeric component, the process comprising
contacting at least one surface region of at least one decellularized tissue having been pierced to form one or more holes with at least one polymer and
permitting said at least one polymer to penetrate into the one or more holes.

78. The process according to claim 77, further comprising at least one step selected from (a) forming holes in a surface region of at least one decellularized tissue; (b) injecting the polymer into the piercings (holes); (c) curing the polymer; (d) permitting said at least one polymer to penetrate into the one or more holes to thereby forming a polymeric sheet on the surface region; (e) associating or fusing two or more constructs; (f) thermal bonding of said at least one decellularized tissue and said at least one polymer by raising the temperature of the polymeric component above either (i) the transition temperature thereof; or, (ii) the melting temperature thereof for semi-crystalline polymers to result, at least partially, in a polymer chain entanglement along at least one region of the polymeric component; (g) selecting the polymeric to be in a form of a layer or a coat of particles, a polymeric sheet, a polymeric film, a polymeric fiber or a polymeric mesh, a gel, a hydrogel or as a liquid or fluidic film and any combination thereof; (h) any combination thereof.

79. The process according to claim 77, wherein at least one of the following is being held true (a) the polymer fully penetrates through the one or more holes; (b) the polymer partially penetrates the one or more holes; (c) the polymer fully penetrates through the one or more holes to form a polymer sheet on both faces of the surface region, to thereby form a construct assembly; and, (d) any combination thereof.

80. A process for manufacturing a construct comprising at least one decellularized tissue and at least one polymeric component, the process comprising
stacking one or more sheets of a decellularized tissue and one or more sheets or segments of a polymeric material to obtain a stacked structure;
forming holes in said stacked structure to form one or more holes in each of the one or more sheets of the tissue and polymeric material, wherein optionally at least a number of said one or more holes are coaxially arranged; and
treating said stacked structure with a liquid polymer to cause said liquid polymer to penetrate into the one or more holes and fuse said sheets to form the construct.

81. The construct according to claim 53, wherein said at least one decellularized tissue is dried before being associated with said polymeric component.

82. The construct according to claim 81, wherein said at least one decellularized tissue is lyophilized.
